(19) United States
(12) Patent Application Publication
(10) Pub. No.: US 2024/0197882 A1
(43) Pub. Date: Jun. 20, 2024

(54) HUMANIZED ANTI-SIALYL-TN GLYCAN ANTIBODIES AND USES THEREOF

(71) Applicant: RAMOT AT TEL-AVIV UNIVERSITY LTD., Tel Aviv (IL)

(72) Inventors: Vered PADLER-KARAVANI, Tel Aviv (IL); Ron AMON, Tel Aviv (IL)

(21) Appl. No.: 18/556,161

(22) PCT Filed: Apr. 13, 2022
(86) PCT No.: PCT/IL2022/050385

§ 371 Date: Oct. 19, 2023

Related U.S. Application Data
(60) Provisional application No. 63/176,895, filed on Apr. 20, 2021.

(51) Int. Cl. A61K 39/00; C07K 14/705; C07K 14/725; C07K 16/32; C12N 5/0783; G01N 33/574

(52) U.S. Cl. CPC A61K 39/464474; A61K 39/4611; A61K 39/4613; A61K 39/4631; C07K 14/7051; C07K 14/70517; C07K 14/70521; C07K 14/70532; C07K 14/70575; C07K 14/70578; C07K 16/32; C12N 5/0636; G01N 33/57492; C07K 2317/24; C07K 2319/03; C07K 2319/30; G01N 2440/38

(57) ABSTRACT

The present invention discloses humanized monoclonal antibodies that specifically bind to STn carbohydrate antigen with high specificity and selectivity, functional fragments of the humanized monoclonal antibodies such as scFv, conjugates and chimeric antigen receptors comprising the humanized monoclonal antibodies or the fragment thereof such as scFv. The invention further provides cells and compositions comprising the antibodies, fragments thereof or CARs as well as their use in diagnostics and treatment of cancer.

### FIELD OF THE INVENTION

The present invention relates to humanized monoclonal antibodies that specifically bind to Sialyl Tn (STn) carbohydrate antigen, fragments thereof, conjugates thereof and CARs comprising the antigen-binding domain of the humanized monoclonal antibodies, as well as to cells comprising said antibodies, fragments or CARs, compositions comprising same and uses thereof.

### BACKGROUND OF THE INVENTION

Aberrant glycosylation is one of the hallmarks of cancer resulting in expression of tumor-associated carbohydrate antigens (TACA) that are overexpressed in many types of cancer such as breast, colorectal, ovary, lung, bladder, etc. As a potential cancer cell marker, these glycans constitute an important target for development of antibodies as therapeutic and diagnostic tools. Currently, there are no clinically used anti-glycan antibodies for cancer treatment, and most therapeutic antibodies are against proteins. Anti-carbohydrate antibodies presumably have low affinity compared to those targeting proteins, and some are of low specificity. Both affinity and specificity are two crucial elements in antibody recognition and are important for clinical applications. There are several methods that allow development and improvement of antibodies with high affinity and specificity. These methods include various display systems using phage, ribosome or yeast. Furthermore, antibodies that are produced in non-human system (e.g. in mice) require antibody humanization to allow their use for human treatment. Antibody humanization involves techniques as framework-homology-based humanization, germline humanization, complementary determining regions (CDR)-homology-based humanization and specificity determining residues grafting (Safdari et al., 2013; Ahmadzadeh et al., 2014; Waldmann, 2019). Nevertheless, selection of mutations that would preserve the original antibody affinity and specificity but with reduced immunogenicity is not at all trivial.

Chimeric antigen receptor (CAR) T cell therapy is one of the most growing fields in cancer therapy. The approval of CD19 directed CAR for treatment of acute lymphoblastic leukemia (ALL) and large B cell lymphoma lead to other trails to apply CD19 CAR for additional B cell malignancies. Moreover, efforts are made to develop novel CARs against tumor associated antigens against various cancers. Despite the great interest in novel CARs, therapy of solid tumors remains a considerable challenge. Administration of CAR against HER2 in human patients resulted in severe toxicity to lung tissue. Yet, additional attempts are made to utilize HER2 directed CARs in different setup. Other novel targets for CARs for solid tumor therapy include IL13Ra2, epidermal growth factor receptor (EGFRvIII), carcino-embryonic antigen (CEA), Mesothelin and others. However, a major challenge in many of these targets is ‘on-target off-tumor’ toxicity. Low level expression of the CAR target in different tissues is sufficient to cause severe side effects and even fatal toxicity.

Sialyl-Tn (STn) is known for decades as tumor associated carbohydrate antigen. STn levels were associated with tumor aggressiveness and resistant to chemotherapy. While STn antigen is expressed in more than 80% of human carcinomas (Julien et al., Biomolecules. 2012 Oct. 11; 2(4):435-66). Nevertheless, despite the abundance of different publications, there are no approved clinical therapeutics that target this antigen. There is an urgent need for development efficient and safe therapies targeting Sialyl-Tn and therefore treating a vast number of different tumors, in particular with CAR T cells immunotherapy.

### SUMMARY OF THE INVENTION

The present invention is based on the results showing that humanized monoclonal antibodies binding to Sialyl Tn (STn) glycan have a decreased immunogenicity on one hand and potent affinity to the STn glycan on the other hand. Amino acid substitutions of framework residues were performed after a thorough examination of the sites and are based not only on similarity to human sequences but also on structural considerations. As it has been shown, the generation of functional humanized antibodies or fragments thereof that have reduced immunogenicity is not an easy task and requires creative thinking. Not all of the generated humanized antibodies indeed provided the desired effect. Possessing lower immunogenicity, the humanized monoclonal antibodies of the present invention as well and their fragments, conjugates and chimeric antigen receptor (CAR) molecules comprising same, avoid the risk of the adverse immune response towards them and therefore are safe for use in humans.

According to one aspect, the present invention provides a humanized monoclonal antibody (mAb) or a fragment or conjugate thereof that specifically binds to Sialyl Tn glycan (STn), wherein the humanized mAb or the fragment comprises an antigen-binding domain comprising a heavy-chain variable domain (VH) and a light-chain variable domain (VL), wherein the VH and the VL each comprises three complementarity determining regions (CDRs) and four framework regions (FR), According to some embodiments, the fragment is a single-chain variable fragment (scFv). The list of all sequences is provided in the sequence table. According to some embodiments, the present invention provides humanized monoclonal antibody (mAb) that specifically binds to Sialyl Tn (STn) glycan or a fragment of the mAb, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH comprises amino acid sequence SEQ ID NO:1 in which at least 9 or at least 12 amino acids in the framework domains are substituted and the VL comprises amino acid sequence SEQ ID NO:2 in which at least 9 or at least 11 amino acids in the framework domains are substituted. According to some embodiments, the present invention provides a humanized monoclonal antibody (mAb) or a fragment thereof that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO:1 in which from 9 to 16 amino acids in the framework regions are substituted and the VL domain comprises amino acid sequence SEQ ID NO:2 in which from 9 to 20 amino acids in the framework regions are substituted. According to some embodiments, the VH-CDRs 1, 2 and 3 comprise or consist of amino acid sequences SEQ ID NOs: 3, 4 and 5, respectively, and the VL-CDRs 1, 2, and 3 comprise or consist of amino acid sequences SEQ ID NOs: 6, 7, and 8, respectively.

According to some embodiments, at least 9 substitutions in VH domain are at positions selected from positions 10, 13, 15, 17, 44, 73, 76, 83, 84, 85 and 113 of SEQ ID NO: 1. Thus, the VH comprises or consists of amino acid sequence SEQ ID NO: 1 in which at least 9 substitutions at FR regions are performed and wherein 9, 10 or 11 substitutions are at positions 10, 13, 15, 17, 44, 73, 76, 83, 84, 85 and 113. According to some embodiments, the VH comprises or consists of amino acid sequence SEQ ID NO: 1 in which all amino acids at positions 10, 13, 15, 17, 44, 73, 76, 83, 84, 85 and 113 of SEQ ID NO: 1 are substituted. According to some embodiments, only amino acids at positions 10, 13, 15, 17, 44, 73, 76, 83, 84, 85 and 113 of SEQ ID NO: 1 are substituted. According to some embodiments, the VH domain comprises or consists of amino acid sequence SEQ ID NO:

According to some embodiments, at least 9 substitutions in the VL domain are at positions selected from positions 1, 10, 11, 13, 18, 19, 21, 22, 39, 41, 42, 57, 69, 70, 71, 77, 99 and 105 of SEQ ID NO: 2. Thus, the VL comprises or consists of amino acid sequence SEQ ID NO: 2 in which at least 9 substitutions at FR regions are performed and wherein 9, 10, 11, 12, 13, 14, 15, 16, 17 or 18 substitutions are at positions 1, 10, 11, 13, 18, 19, 21, 22, 39, 41, 42, 57, 69, 70, 71, 77, 99 and 105. According to some embodiments, the VL comprises or consists of amino acid sequence SEQ ID NO: 2 in which all amino acids at positions 1, 10, 11, 13, 18, 39, 41, 42, 57, 77, 99 and 105 are substituted. According to some embodiment, the VL comprises amino acid sequence SEQ ID NO: 2 wherein all and only amino acids at positions 1, 10, 11, 13, 18, 39, 41, 42, 57, 77, 99 and 105 of SEQ ID NO: 2 are substituted. According to other embodiments, the VL comprises amino acid sequence SEQ ID NO: 2 in which all amino acids at positions 1, 10, 11, 13, 18, 19, 21, 22, 39, 41, 42, 57, 69, 70, 71, 77, 99 and 105 are substituted. According to some embodiments, the VL comprises amino acid sequence SEQ ID NO: 2 wherein all and only amino acids at positions 1, 10, 11, 13, 18, 19, 21, 22, 39, 41, 42, 57, 69, 70, 71, 77, 99 and 105 of SEQ ID NO: 2 are substituted. According to some embodiments, the VL domain comprises or consists of amino acid sequence SEQ ID NO: 29. According to some embodiments, the VL domain comprises or consists of amino acid sequence SEQ ID NO: 30.

According to some embodiments, the present invention provides a humanized mAb or the fragment thereof wherein the VH and VL domains comprise or consist of amino acid sequence SEQ ID NO: 28 and 29, respectively. According to some embodiments, the present invention provides a humanized mAb or the fragment thereof wherein the VH and VL domains comprises or consists of amino acid sequence SEQ ID NO: 28 and 30, respectively.

According to any one of the above aspects and embodiments, the humanized mAb or the fragment binds STn glycan with an equilibrium dissociation constant (K_{D}) from about 0.1 to about 30 nM or from 0.02 to 0.5 nM.

According to some embodiments, the fragment of the humanized antibody of the present invention is a single chain variable fragment (scFv). According to some embodiments, the scFv comprises an amino acid sequence selected from SEQ ID NO: 31 and 32.

According to one aspect, the present intention provides a conjugate of the humanized mAb or fragment thereof of the present invention. According to some embodiments, the conjugates comprises an anti-cancer moiety. According to other embodiments, the conjugate comprises a tag or a label.

According to another aspect, the present invention provides a chimeric antigen receptor (CAR) comprising the humanized mAb or the fragment of the present invention. According to some embodiments, the CAR comprises a fragment of the present invention. According to some embodiment, the fragment is a single-chain variable fragment (scFv). According to some embodiments, scFv comprises an antigen-binding domain comprising the VH and VL domains of the present invention. According to some embodiments, the scFv comprises the VH and VL domains comprising or consisting of amino acid sequences SEQ ID NO: 28 and 29, respectively. According to some embodiments, the scFv comprises the VH and VL domains comprising or consisting of amino acid sequences 28 and 30, respectively. According to some embodiments, the scFv comprises an amino acid sequence selected from SEQ ID NO: 31 and 32. According to some embodiments, the CAR comprises a transmembrane domain, a costimulatory domain and an activation domain. According to some embodiments, the transmembrane domain is the transmembrane domain of a receptor selected from CD28 and CD8, or an analog thereof having at least 85% amino acid identity to the original sequence, and/or the costimulatory domain is selected from a costimulatory domain of a protein selected from CD28, 4-1BB, OX40, iCOS, CD27, CD80, and CD70, an analog thereof having at least 85% amino acid identity to the original sequence and any combination thereof, and/or the activation domain is selected from FcRγ and CD3-5 activation domains. According to some embodiments, the CAR comprises a scFv sequence comprising the binding site of the humanized monoclonal antibody that binds STn a TM domain and a costimulatory domain of CD28, and an activation domain selected from FcRγ and CD3-ζ activation domains. According to specific embodiments, the CAR comprises a scFv comprising amino acid sequence selected from SEQ ID NO: 31 and 32, a TM domain selected from a TM domain of a receptor selected from CD28 and CD8, a costimulatory domain selected from CD28, 4-1BB, OX40, iCOS, CD27, CD80, CD70, an analog thereof and any combination thereof, and an activation domain selected from FcRγ and CD3-ζ activation domain. According to some embodiments, the CAR comprises a tag such as a strep tag. According to some embodiments, the CAR comprises or consists of an amino acid sequence selected from SEQ ID NO: 41, 42, 48 and 49.

According to another aspect, the present invention provides a nucleic acid molecule encoding at least one chain of the humanized monoclonal antibody or fragment thereof or the CAR of the present invention. According to some embodiments, the nucleic acid molecule encoding at least one amino acid sequence selected from SEQ ID NO: 28, 29, 30, 31, 32, a combination of SEQ ID NO: 28 and 29, and a combination of SEQ ID NO: 28 and 30. According to some embodiments, the nucleic acid molecule comprises one or more of the nucleic acid sequence selected from SEQ ID NO: 33, 34, 35, 45, 36, 37, 41, 42, 50, and 51.

According to another aspect, the present invention provides a nucleic acid construct comprising the nucleic acid of the present invention, operably linked to a promoter.

According to another aspect, the present invention provides a vector comprising the nucleic acid molecule, or the nucleic acid construct of the present invention.

According to yet another aspect, the present invention provides a cell comprising the humanized monoclonal antibody or the antibody fragment, the CAR, the nucleic acid molecule, the nucleic acid construct or the vector of the present invention. According to some embodiments, the cell is a mammalian cell. According to some embodiments, the cell is a lymphocyte. According to some embodiments, the cell is a T-cells. According to some embodiments, the cell is a T-cell comprising the CAR of the present invention. According to some embodiments, the cells are T-cells comprising the nucleic acid molecule of the present invention and expressing or are capable of expressing the CAR of the present invention. According to some embodiments, the cell, such as T-cells comprise, express or are capable of expressing the CAR comprising an amino acid sequence selected from SEQ ID NO: 28, 29, 30, 31, 32, a combination of SEQ ID NO: 28 and 29, and a combination of SEQ ID NO: 28 and 30. According to some embodiments, the cell, such as T-cells comprise a nucleic acid molecule sequence selected from SEQ ID NO: 33, 34, 35, 45, 36, 37, 41, 42, 50, 51 and a combination thereof.

According to some embodiments, a lymphocyte engineered to express the CAR described herein is provided. According to some embodiments, a T cell engineered to express the CAR described herein is provided. According to additional embodiments, an NK cell engineered to express the CAR described herein is provided.

According to some embodiment, the cell is capable of producing the humanized monoclonal antibody or the antibody fragment of the present invention.

According to some aspects, the present invention provides a composition comprising the humanized monoclonal antibodies or antibody fragments of the present invention, the conjugate of the present invention, the CAR or the cells of the present invention and a carrier. According to some embodiments, the composition is a pharmaceutical composition and the carrier is a pharmaceutically acceptable carrier. According to some embodiments, the present invention provides a pharmaceutical composition comprising the CAR of the present invention, and a pharmaceutically acceptable carrier. According to some embodiments, the present invention provides a pharmaceutical composition comprising a plurality of cells of the present invention, and a pharmaceutically acceptable carrier. According to some embodiments, the cells are T-cells. According to some embodiments, the T-cells comprise the CAR of the present invention.
a. According to some embodiments, the pharmaceutical composition of the present invention is for use in treating cancer. According to some embodiments, the pharmaceutical composition comprising a plurality of T-cell comprising the CAR of the present invention is for use in treating cancer. According to some embodiments, the cancer is selected from carcinoma and lymphoma. According to some embodiments, the cancer is selected from endometrial carcinoma, breast cancer, ovarian carcinoma, prostate adenocarcinoma, seminoma, diffuse type gastric adenocarcinoma, pancreatic and colon adenocarcinomas, lung adenocarcinoma and mantle cell lymphoma. According to some embodiments, the cancer is selected from breast, lung, ovarian, pancreatic, colon, stomach, oropharyngeal cancer, squamous cell carcinoma, head and neck and gallbladder cancer. According to some particular embodiments, the cancer is selected from lung adenocarcinoma, pancreatic adenocarcinoma, colon adenocarcinoma, Her-2 negative breast carcinoma and pharynx squamous cell carcinoma.

According to yet another aspect, the present invention provides a method for treating cancer in a subject in need thereof comprising administering to said subject a therapeutically effective amount of the monoclonal antibodies or fragments thereof, the conjugate of the present invention, the CAR, the T cells or the pharmaceutical composition of the present invention.

### BRIEF DESCRIPTION OF THE DRAWINGS

FIG. 1 shows a multiple sequence alignment (MSA) of amino acid sequences of the mouse-derived RA0 antibody (mRA0) and the humanized clones HuRA0-V1, HuRA0-V2, HuRA0-V7 and HuRA0-V8 antibodies, respectively, separately showing the VH (FIG. 1A) and VL (FIG. 1B) amino acid sequence alignment. The VL fragment of HuRA0-V1 and HuRA0-V2 is the same one, while their VH fragments are different. The VH fragment of HuRA0-V7 and HuRA0-V8 is the same one, while their VL fragments are different.

FIG. 2 shows binding of mRA0, HuRA0-V7 or HuRA0-V8 to their specific antigen (STn) or to their non-specific antigen (Tn), as examined by FACS. For this purpose, yeast cells with surface expression of scFv fragments of mRA0, HuRA0-V7 or HuRA0-V8 were incubated with either 0.5 μM STn-PAA-Biotin, 0.5 μM Tn-PAA-Biotin or FACS buffer for negative control, then antibody binding detected with secondary detection APC-streptavidin, and measured by CytoFLEX flow cytometry.

FIG. 3 shows the specificity of the full-length antibodies ChRA0-IgG (FIG. 3A), HuRA0-V7-IgG (FIG. 3B), or HuRA0-V8-IgG (FIG. 3C) examined by ELISA inhibition assay against coated STn-PAA-biotin, after pre-incubation of the antibody with specific (STn; STn-PAA-Biotin) or non-specific glycans (Tn or SLea; respectively with Tn-PAA-Biotin or SLea-PAA-Biotin)**** p<0.0001.

FIG. 4 shows the binding of full-length antibodies ChRA0-IgG (FIG. 4A), HuRA0-V7-IgG (FIG. 4B), or HuRA0-V8-IgG (FIG. 4C) against diverse glycans. The binding was examined by a sialoglycan microarray (List of glycans in Table 1).

FIG. 5 shows the binding of humanized full-length antibodies to cancer cells compared with the mouse-derived clone (HuRA0-V7-IgG, HuRA0-V8-IgG, ChRA0-IgG). The binding of IgGs to STn-expressing B16F10 mouse melanoma cancer cell line was examined by FACS at 20 ng/μl. Representative of three independent experiments is provided.

FIGS. 6A-C show cancer cell binding specificity of the antibodies, as demonstrated by the treatment of B16F10 cells with Arthrobacter Urcafaciens Sialidasc (AUS) that abrogated binding of ChRA0-IgG (FIG. 6A), HuRA0-V7-IgG (FIG. 6B), or HuRA0-V8-IgG (FIG. 6C) antibodies to the cells.

FIG. 7 shows reduced immunogenicity of humanized antibodies. Binding of pooled human IgG (pre-cleared of anti-yeast reactivity; yeast-purified IVIg) at 50 ng/μl, 100 ng/μl and 200 ng/μl to scFv-HuRA0-V7 or scFv-HuRA0-V8 yeast cells compared to scFv-mRA0 yeast cells. Cells were first gated for scFv presenting cells by the AF488 fluorescence (stained by mouse-anti-c-Myc followed by Alexa-Fluor-488-goat-anti-mouse IgG1) (FIG. 7A). Positive IVIg binding on the gated scFv presenting cells was then determined by a double-positive signal of scFv presentation by c-myc labeling (AF488) and by binding of IVIg (Cy3; IVIg followed by Cy3-anti-human IgG Fc specific) (FIG. 7B). Then, IVIg-positive cells and IVIg-negative cells were separately gated (FIG. 7C exemplified gating for mRA0 cells labeled with IVIg at 100 ng/μl), and in each IVIg concentration the percent of IVIg-positive cells was divided by the percent of IVIg-negative cells. In each clone, the percentage ratio of (% IVIg-positive cells/% IVIg-negative cells) calculated for the three IVIg concentrations (50, 100 and 200 ng/μl) was averaged. This analysis revealed that the percentage ratio was highest in mNative and it was reduced in the humanized clones: mRA0 (32.9±2%), HuRA0-V7 (25.4±0.7%), HuRA0-V8 (21.7±0.6%). Then, the ratios were normalized to mRA0 yeast cells IVIg percent ratio, which was referred as the maximal signal (100%) (FIG. 7D). These data show reduced immunogenicity of about 25% of HuRA0-V7 scFv in comparison to mRA0, and a reduction of about 36% in immunogenicity of HuRA0-V8 compared with mRA0 (FIG. 7D). Representative of two independent experiments. One way ANOVA, Tukey, ** p<0.01, *** p<0.001, **** p<0.0001.

### DETAILED DESCRIPTION OF THE INVENTION

According to one aspect, the present invention provides a humanized monoclonal antibody (mAb) or a fragment thereof that specifically binds to Sialyl Tn glycan (STn), wherein the mAb or the fragment comprises an antigen-binding domain comprising a heavy-chain variable domain (VH) and a light-chain variable domain (VL), wherein the VH comprises amino acid sequence SEQ ID NO: 1 in which 9 or more amino acid residues in the framework regions are substituted and the VL comprises amino acid sequence SEQ ID NO: 2 in which 9 or more amino acid residues in the framework regions are substituted. According to some embodiments, the present invention provides a humanized monoclonal antibody (mAb) that specifically binds to Sialyl Tn glycan (STn), comprising an antigen-binding domain comprising a VH having amino acid sequence SEQ ID NO: 1 in which from 9 to 35 amino acid residues in the framework regions are substituted and a VL having amino acid sequence SEQ ID NO: 2 in which from 9 to 35 amino acid residues in the framework regions are substituted. According to other embodiments, the present invention provides a fragment of the humanized monoclonal antibody (mAb) that specifically binds to Sialyl Tn glycan (STn), comprising an antigen-binding domain comprising a VH having amino acid sequence SEQ ID NO: 1 in which from 9 to 35 amino acid residues in the framework regions are substituted and a VL having amino acid sequence SEQ ID NO: 2 in which from 9 to 35 amino acid residues in the framework regions are substituted.

According to some embodiments, the present invention provides a humanized monoclonal antibody (mAb) that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO: 1 in which from 9 to 16 amino acids in the framework regions are substituted and the VL domain comprises amino acid sequence SEQ ID NO:2 in which from 9 to 20 amino acids in the framework regions are substituted. According to some embodiments, the present invention provides a fragment of the humanized monoclonal antibody (mAb) that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO:1 in which from 9 to 16 amino acids in the framework regions are substituted and the VL domain comprises amino acid sequence SEQ ID NO:2 in which from 9 to 20 amino acids in the framework regions are substituted.

According to any one of the above embodiments, each VH and VL comprises three complementarity determining regions (CDRs) and four framework regions (FR).

The terms “antibody”, “antibodies” and “Ab” are used here interchangeably in their broadest sense and includes monoclonal antibodies (including full length or intact monoclonal antibodies), polyclonal antibodies, multivalent antibodies, multi-specific antibodies (e.g., bi-specific antibodies), and antibody fragment long enough to exhibit the desired biological activity.

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (Fragment crystalline) domains. The term “antigen-binding portion”, “antigen-binding region”, “antigen-binding site”, “antigen-binding domain” and “ABD” are used herein interchangeably and refer to one or more fragments of an antibody that retain the ability to specifically bind to an antigen. The antigen-binding domains, Fab, include regions where the polypeptide sequence varies. The term F (ab′)₂ represents two Fab′ arms linked together by disulfide bonds. The central axis of the antibody is termed the Fc fragment. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1). The variable domains of each pair of light and heavy chains form the antigen-binding site. The domains of the light and heavy chains have the same general structure and each domain comprises four framework regions, whose sequences are relatively conserved, joined by three hyper-variable domains known as complementarity determining regions (CDRs). These domains contribute to specificity and affinity of the antigen-binding site. The isotype of the heavy chain (gamma, alpha, delta, epsilon or mu) determines immunoglobulin class (IgG, IgA, IgD, IgE or IgM, respectively). The light chain is either of two isotypes (kappa (κ) or lambda (λ)) found in all antibody classes. The term “paratope” refers to the antigen-binding site of an antibody or fragment thereof.

The terms “monoclonal antibody” and “mAb” are used herein interchangeably and refer to an antibody obtained from a population of substantially homogeneous antibody, i.e., the individual antibody comprising the population are identical except for possible naturally occurring mutations that may be present in minor amounts.

Monoclonal antibodies (mAbs) are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method, mAbs may be obtained by methods known to those skilled in the art. For example, the monoclonal antibodies to be used in accordance with the present invention may be made by the Hybridoma method or may also be isolated from phage antibody libraries.

The term “humanized antibodies” refers to antibodies from non-human species (e.g. murine antibodies) whose amino acid sequences have been modified to increase their similarity to antibody variants produced naturally in humans. The process of “humanization” is usually applied to monoclonal antibodies developed for administration to humans, and performed when the process of developing a specific antibody involves generation in a non-human immune system (such as in mice). The protein sequences of antibodies produced in this way are distinct from antibodies occurring naturally in humans, and are therefore immunogenic when administered to human patients. Humanized antibodies are considered distinct from chimeric antibodies, which have protein sequences similar to human antibodies, but carry large stretches of non-human protein.

The terms “fragment”, “functional fragment” and “antibody fragment” are used herein interchangeably and refer to only a portion of an intact antibody, generally including an antigen-binding site of the intact antibody and thus retaining the ability to bind antigen. The term refers to the antibody as well as to the analog or variant of said antibody. The antibody fragment according to the teaching of the present invention is a functional fragment, i.e. preserves the function of the intact antibody. Examples of antibody fragment encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 1988, 242, 423-426; and Huston et al., PNAS (USA) 1988, 85, 5879-5883); (x) “diabodies” with two antigen-binding sites, comprising a heavy chain variable domain (VH) connected to a light chain variable domain (VL) in the same polypeptide chain (sec, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen-binding regions. According to some embodiments, the functional fragment is an scFv.

The terms “light chain variable region”, “VL” and “VL” are used herein interchangeably and refer to a light chain variable region of an antibody capable of binding to STn glycan. The terms “heavy chain variable region”, “VH” and “VH” are used herein interchangeably and refer to a heavy chain variable region of an antibody capable of binding to STn glycan.

As used herein, the term “CDR” refers to the complementarity determining region within antibody variable sequences. There are three CDRs in each one of the variable regions of the heavy chain and the light chain, which are designated CDR1, CDR2 and CDR3 (or specifically VH CDR1, VH CDR2, VH CDR3, VL CDR1, VL CDR2, and VL CDR3), for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. Still other CDR boundary definitions may not strictly follow one of the known systems, but will nonetheless overlap with the Kabat CDRs, although they may be shortened or lengthened in light of prediction or experimental findings that particular residues or groups of residues or even entire CDRs do not significantly impact antigen binding. Determination of CDR sequences from antibody heavy and light chain variable regions can be made according to any method known in the art, including but not limited to the methods known as KABAT, Chothia and IMGT. The selected set of CDRs may include sequences identified by more than one method, namely, some CDR sequences may be determined using KABAT and some using IMGT. According to one embodiment, the CDRs are defined using KABAT method.

As used herein, the terms “framework”, “framework region” or “framework sequence” refer to the remaining sequences of a variable region minus the CDRs. Because the exact definition of a CDR sequence can be determined by different systems, the meaning of a framework sequence is subject to correspondingly different interpretations. The six CDRs also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

According to some embodiments, the antibody fragment is a single chain variable fragment being a composite polypeptide having antigen-binding capabilities and comprising amino acid sequences homologous or analogous to the variable regions of an immunoglobulin light and heavy chain i.e. linked V_{H}-V_{L}, V_{L}-V_{H} or single chain Fv (scFv).

According to some embodiments, the terms “antibody” or “antibodies” collectively refer to intact antibodies, i.e. humanized monoclonal antibodies (mAbs) and analogs thereof, as well as proteolytic fragments thereof, such as the Fab or F(ab′)₂ fragments and scFv.

The terms “binds specifically” or “specific for” with respect to an antigen-binding domain of an antibody or of a fragment thereof refers to an antigen-binding domain that recognizes and binds to a specific antigen, but does not substantially recognize or bind other molecules, e.g. in a sample or in vivo. The term contemplates that the antigen-binding domain binds to its antigen with high affinity and binds other antigens with low affinity. An antigen-binding domain that binds specifically to an antigen from one species may bind also to that antigen from another species. This cross-species reactivity is not contrary to the definition of that antigen-binding domain as specific. The term “K_{D}”, as used herein, is intended to refer to the dissociation constant of a particular antibody-antigen interaction. K_{D} is calculated as kₐ/k_{d}. The term “kₒₙ” or “kₐ”, as used herein, is intended to refer to the on-rate constant for association of an antibody to the antigen to form the antibody/antigen complex. The term “k_{off}” or “kₐ”, as used herein, is intended to refer to the off-rate constant for dissociation of an antibody from the antibody/antigen complex.

The terms “Sialyl Tn glycan”, and “STn” are used herein interchangeably and refer to Neu5Acα2-6GalNAcαO(CH2)₂CH₂NH₂ disaccharide carbohydrate, and having the structure as presented in Structure I.

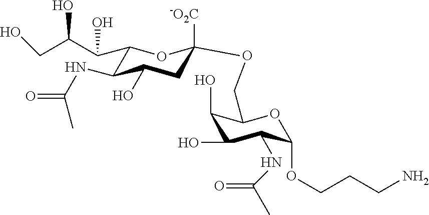

### Structure I.

The term “non-conservative substitutions”, as used herein shall mean the substitution of one amino acid by another which has different properties (i.e., charge, polarity, hydrophobicity, structure). Examples of the non-conservative substitution include substitution of a hydrophobic residue such as isoleucine, valine, leucine, alanine, phenylalanine, tyrosine, tryptophan or methionine for a polar or charged amino acid residue such as lysine, arginine, glutamine, asparagine, aspartate, glutamate, histidine serine, threonine, or cysteine. Likewise, the present disclosure contemplates the substitution of a charged amino acid such as lysine, arginine, histidine, aspartate and glutamate for an uncharged residue including, but not limited to serine, threonine, asparagine, glutamine, or glycine. In certain embodiments, non-conservative substitutions include substitution of an uncharged, hydrophobic amino acid such as leucine with a charged amino acid, such as aspartic acid, lysine, arginine, or glutamate.

The term “conservative substitution” as used herein denotes the replacement of an amino acid residue by another, without altering the overall conformation and biological activity of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

According to some embodiments, at least 9 substitutions in VH domain are at positions selected from positions 10, 13, 15, 17, 44, 73, 76, 83, 84, 85 and 113 of SEQ ID NO: 1. Thus, according to some embodiments, the VH domain comprises an amino acid sequence SEQ ID NO:1 in which from 9 to 16 amino acids in the framework regions are substituted, wherein at least 9 substitutions are at positions selected from positions 10, 13, 15, 17, 44, 73, 76, 83, 84, 85 and 113 of SEQ ID NO: 1. According to some embodiments, the VH domain comprises an amino acid sequence SEQ ID NO:1 in which from 9 to 13 amino acids in the framework regions are substituted, wherein at least 9 are at positions selected from positions 10, 13, 15, 17, 44, 73, 76, 83, 84, 85 and 113 of SEQ ID NO: 1. According to some embodiments, none of the substitutions is made at positions 3, 5, 19, 20, 24, 68, 70, 78, 81, 82, 86, 87 and 88 SEQ ID NO: 1.

According to some embodiments, the VH domain comprises an amino acid sequence SEQ ID NO:1 in which all amino acids at positions 10, 13, 15, 17, 44, 73, 76, 83, 84, 85 and 113 are substituted. According to some embodiments, the VH domain consists of an amino acid sequence SEQ ID NO: 1 in which all amino acids at positions 10, 13, 15, 17, 44, 73, 76, 83, 84, 85, and 113 are substituted. According to some embodiments, the substitutions may be conservative or non-conservative. According to some embodiments, the amino acid at positions 10, 44 and 85 are substituted for a hydrophobic amino acid, e.g. Ala, Val, Leu, Ile or Met. According to some embodiments, the amino acid at position 13 is substituted for a positively charged amino acid, such as Lys and Arg. According to some embodiments, the amino acid at positions 15, 17, 73, 76, 83, 84, and 113 are substituted for a polar amino acid such as Ser, Thr, Gln and Asn. According to some embodiments, the VH domain comprises an amino acid sequence SEQ ID NO:1 in which amino acids at positions 10, 44 and 85 are substituted for a hydrophobic amino acid selected from Ala, Val, Leu, Ile and Met, the amino acid at position 13 is substituted for a positively charged amino acid selected from Lys and Arg, and the amino acids at positions 15, 17, 73, 76, 83, 84 and 113 are substituted for a polar amino acid selected from Ser, Thr, Gln and Asn. According to some embodiments, the VH domain consists of an amino acid sequence SEQ ID NO:1 in which amino acids at positions 10, 44 and 85 are substituted for a hydrophobic amino acid selected from Ala, Val, Leu, Ile and Met, the amino acid at position 13 is substituted for a positively charged amino acid selected from Lys and Arg, and the amino acids at positions 15, 17, 73, 76, 83, 84 and 113 are substituted for a polar amino acid selected from Ser, Thr, Gln and Asn. According to some embodiments, the VH domain comprises the amino acid sequence SEQ ID NO: 28. According to some embodiments, the VH domain consists of the amino acid sequence SEQ ID NO: 28.

According to some embodiments, at least 9 substitutions in VL domain are at positions selected from positions 1, 10, 11, 13, 18, 19, 21, 22, 39, 41, 42, 57, 69, 70, 71, 77, 99 and 105 of SEQ ID NO: 2. Thus, according to some embodiments, the VL domain comprises an amino acid sequence SEQ ID NO:2 in which from 9 to 25 amino acids in the framework regions are substituted, wherein at least 9 substitutions are at positions selected from positions 1, 10, 11, 13, 18, 19, 21, 22, 39, 41, 42, 57, 69, 70, 71, 77, 99 and 105 of SEQ ID NO: 2. According to some embodiments, the VL domain comprises an amino acid sequence SEQ ID NO:2 in which from 9 to 20 amino acids in the framework regions are substituted, wherein at least 9 are at positions selected from positions 1, 10, 11, 13, 18, 19, 21, 22, 39, 41, 42, 57, 69, 70, 71, 77, 99 and 105 of SEQ ID NO: 2. According to some embodiments, none of the substitutions is made at positions 44, 45, 46, 79, 82 and 84 of SEQ ID NO: 2.

According to some embodiments, the VL domain comprises an amino acid sequence SEQ ID NO:2 in which all amino acids at positions 1, 10, 11, 13, 18, 39, 41, 42, 57, 77, 99 and 105 are substituted. According to some embodiments, the VL domain consists of an amino acid sequence SEQ ID NO:2 in which all amino acids at positions 1, 10, 11, 13, 18, 39, 41, 42, 57, 77, 99 and 105 are substituted. According to some embodiments, the substitutions may be conservative or non-conservative. According to some embodiments, the amino acid at positions 11, 13, 42, 57, 77, and 105 are substituted for a hydrophobic amino acid, e.g. Ala, Val, Leu, Ile or Met. According to some embodiments, the amino acid at position 18 is substituted for a positively charged amino acid, such as Lys and Arg. According to some embodiments, the amino acid at position 39 is substituted for Pro. According to some embodiments, the amino acid at position 1 is substituted for a negatively charged amino acid such as Asp or Glu. According to some embodiments, the amino acid at positions 10, 41 and 99 are substituted for a polar amino acid such as Ser, Thr, Gln and Asn. According to some embodiments, the VL domain comprises an amino acid sequence SEQ ID NO:2 in which the amino acid at position 1 is substituted for a negatively charged amino acid selected from Asp and Glu, amino acids at positions 11, 13, 42, 57, 77, and 105 and are substituted for a hydrophobic amino acid selected from Ala, Val, Leu, Ile and Met, the amino acid at position 18 is substituted for a positively charged amino acid selected from Lys and Arg, the amino acids at positions 10, 41 and 99 are substituted for a polar amino acid selected from Ser, Thr, Gln and Asn, and the amino acid at position 39 is substituted for Pro. According to some embodiments, the VL domain consists of an amino acid sequence SEQ ID NO:2 in which the amino acid at position 1 is substituted for a negatively charged amino acid selected from Asp and Glu, amino acids at positions 11, 13, 42, 57, 77, and 105 and are substituted for a hydrophobic amino acid selected from Ala, Val, Leu, Ile and Met, the amino acid at position 18 is substituted for a positively charged amino acid selected from Lys and Arg, the amino acids at positions 10, 41 and 99 are substituted for a polar amino acid selected from Ser, Thr, Gln and Asn, and the amino acid at position 39 is substituted for Pro. According to some embodiments, the VL domain comprises the amino acid sequence SEQ ID NO: 29. According to some embodiments, the VH domain consists of amino acid sequence SEQ ID NO: 29.

According to some embodiments, the VL domain comprises an amino acid sequence SEQ ID NO:2 in which all amino acids at positions 1, 10, 11, 13, 18, 19, 21, 22, 39, 41, 42, 57, 69, 70, 71, 77, 99 and 105 are substituted. According to some embodiments, the VL domain comprises or consists of an amino acid sequence SEQ ID NO:2 in which the amino acid at position 1 is substituted for a negatively charged amino acid selected from Asp and Glu, amino acids at positions 11, 13, 19, 21, 42, 57, 77, and 105 and are substituted for a hydrophobic amino acid selected from Ala, Val, Leu, Ile and Met, the amino acid at position 18 is substituted for a positively charged amino acid selected from Lys and Arg, the amino acids at positions 10, 22, 41, 71 and 99 are substituted for a polar amino acid selected from Ser, Thr, Gln and Asn, the amino acid at position 69 is substituted for a negatively charged amino acid, such as Asp and Glu, the amino acid at position 70 is substituted for a bulky hydrophobic amino acid, such as Phe or Trp, and the amino acid at position 39 is substituted for Pro. According to some embodiments, the VL domain comprises the amino acid sequence SEQ ID NO: 30. According to some embodiments, the VH domain consists of amino acid sequence SEQ ID NO: 30.

According to some embodiments, the VH domain comprises an amino acid sequence SEQ ID NO: 1 in which all amino acids at positions 10, 13, 15, 17, 44, 73, 76, 83, 84, 85 and 113 are substituted, and the VL domain comprises an amino acid sequence SEQ ID NO:2 in which all amino acids at positions 1, 10, 11, 13, 18, 39, 41, 42, 57, 77, 99 and 105 and optionally at positions 19, 21, 22, 69, 70, and 71 are substituted.

According to some embodiments, the VH domain comprises or consist of an amino acid sequence SEQ ID NO:1 in which amino acids at positions 10, 44 and 85 and are substituted for a hydrophobic amino acid selected from Ala, Val, Leu, Ile and Met, the amino acid at position 13 is substituted for a positively charged amino acid selected from Lys and Arg, and the amino acids at positions 15, 17, 73, 76, 83, 84 and 113 are substituted for a polar amino acid selected from Ser, Thr, Gln and Asn, and the VL domain comprises or consists of an amino acid sequence SEQ ID NO:2 in which the amino acid at position 1 is substituted for a negatively charged amino acid selected from Asp and Glu, amino acids at positions 11, 13, 42, 57, 77, and 105 and are substituted for a hydrophobic amino acid selected from Ala, Val, Leu, Ile and Met, the amino acid at position 18 is substituted for a positively charged amino acid selected from Lys and Arg, the amino acids at positions 10, 41 and 99 are substituted for a polar amino acid selected from Ser, Thr, Gln and Asn, and the amino acid at position 39 is substituted with Pro.

According to some embodiments, the VH domain comprises or consist of an amino acid sequence SEQ ID NO:1 in which amino acids at positions 10, 44 and 85 and are substituted for a hydrophobic amino acid selected from Ala, Val, Leu, Ile and Met, the amino acid at position 13 is substituted for a positively charged amino acid selected from Lys and Arg, and the amino acids at positions 15, 17, 73, 76, 83, 84 and 113 are substituted for a polar amino acid selected from Ser, Thr, Gln and Asn and the VL domain comprises or consists of an amino acid sequence SEQ ID NO:2 in which the amino acid at position 1 is substituted for a negatively charged amino acid selected from Asp and Glu, amino acids at positions 11, 13, 19, 21, 42, 57, 77, and 105 and are substituted for a hydrophobic amino acid selected from Ala, Val, Leu, Ile and Met, the amino acid at position 18 is substituted for a positively charged amino acid selected from Lys and Arg, the amino acids at positions 10, 22, 41, 71 and 99 are substituted for a polar amino acid selected from Ser, Thr, Gln and Asn, the amino acid at position 69 is substituted for a negatively charged amino acid such as Asp and Glu, the amino acid at position 70 is substituted for a bulky hydrophobic amino acid such as Phe or Trp, and the amino acid at position 39 is substituted with Pro.

According to some embodiments, the present invention provides humanized monoclonal antibody (mAb) or a fragment thereof that specifically binds to Sialyl Tn (STn) glycan wherein the VH domain comprises the amino acid sequence SEQ ID NO: 28 the VL domain comprises the amino acid sequence SEQ ID NO: 29. According to some embodiments, the VH domain consists of amino acid sequence SEQ ID NO: 28 and the VL domain consists of the amino acid sequence SEQ ID NO: 29.

According to some embodiments, the present invention provides a humanized monoclonal antibody (mAb) or a fragment thereof that specifically binds to Sialyl Tn (STn) glycan wherein the VH domain comprises the amino acid sequence SEQ ID NO: 28 the VL domain comprises the amino acid sequence SEQ ID NO: 30. According to some embodiments, the VH domain consists of amino acid sequence SEQ ID NO: 28 and the VL domain consists of the amino acid sequence SEQ ID NO: 30.

According to any one of the above embodiments, the functional fragment is an scFv. Thus, according to some embodiments, the present invention provides a single-chain variable fragment comprising an antigen-binding domain comprising a heavy-chain variable domain (VH) and a light-chain variable domain (VL), wherein the VH and the VL each comprises three complementarity determining regions (CDRs) and four framework regions (FR). According to some embodiments, the VH and the VL domains of the CAR of the present invention are linked by a spacer to form a single chain variable fragment (scFv). According to some embodiments, the present invention provides a scFv comprising VH and VL, wherein the VH comprises amino acid sequence SEQ ID NO:1 in which at least 9 amino acids in the framework domains are substituted and the VL comprises amino acid sequence SEQ ID NO:2 in which at least 9 amino acids in the framework domains are substituted. The terms “linker” or “spacer” in the context of the scFv or CAR refers to any peptide capable of connecting two domains of the ABD or CAR or two distinguishable sections of the CAR such as variable domains with its length depending on the kinds of variable domains to be connected. According to any one of the above embodiments, the VL and VH domains in the scFv may be placed in any order, such as N′-VH-VL-C′ or N′-VL-VH-C′. The VH and VL domains may be linked by a linker. According to some embodiments, the linker comprises an amino acid sequence SEQ ID NO: 9. According to some embodiments, the linker comprises an amino acid sequence being a repetition of amino acid sequence SEQ ID NO: 9, e.g. 2, 3, 4, 5, or 6 repetitions. According to some embodiments, the linker comprises amino acid sequence SEQ ID NO: 10.

According to some embodiments, the scFv comprises the VH and VL domains as described above.

According to some embodiments, the present invention provides an scFv that specifically binds to Sialyl Tn (STn) glycan wherein the VH domain comprises the amino acid sequence SEQ ID NO: 28 the VL domain comprises the amino acid sequence SEQ ID NO: 29. According to some embodiments, the VH domain consists of amino acid sequence SEQ ID NO: 28 and the VL domain consists of the amino acid sequence SEQ ID NO: 29.

According to some embodiments, the present invention provides a scFv that specifically binds to Sialyl Tn (STn) glycan wherein the VH domain comprises the amino acid sequence SEQ ID NO: 28 the VL domain comprises the amino acid sequence SEQ ID NO: 30. According to some embodiments, the VH domain consists of amino acid sequence SEQ ID NO: 28 and the VL domain consists of the amino acid sequence SEQ ID NO: 30.

According to some embodiments, the scFv comprises amino acid sequence SEQ ID NO: 31. According to some embodiments, the scFv consists of amino acid sequence SEQ ID NO: 31.

According to some embodiments, the scFv comprises amino acid sequence SEQ ID NO 32. According to some embodiments, the scFv comprises amino acid sequence SEQ ID NO 32.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” also encompass the meaning of “consisting of” and “consisting essentially of”, and may be substituted by these terms.

According to any one of the aspects and embodiments of the invention, the terms “comprising amino acid sequence set forth in SEQ ID NO: X”, “comprising SEQ ID NO: X” and “having SEQ ID NO: X” are used herein interchangeably. The terms “consisting of the amino acid sequence set forth in SEQ ID NO: X”, “consisting of SEQ ID NO: X” and “of SEQ ID NO: X” are used herein interchangeably.

The same rule holds for nucleic acid sequence. Thus, the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably.

According to any one of the above embodiments, the humanized mAb or the fragment of the present invention binds STn glycan with an equilibrium dissociation constant (K_{D}) of about 0.01 to 100 nM. According to one embodiment, the mAb or the fragment of the present invention binds STn glycan with an equilibrium dissociation constant (K_{D}) of about 0.05 to 80 nM, about 0.075 to 60 nM. According to one embodiment, the mAb or the fragment of the present invention binds STn glycan with an equilibrium dissociation constant (K_{D}) of about 0.1 to 30 nM. According to some embodiment, the mAb or the fragment of the present invention binds STn glycan with an equilibrium dissociation constant (K_{D}) of about 0.1 to 20 nM. According to one embodiment, the humanized mAb or the fragment of the present invention binds STn glycan with an equilibrium dissociation constant (K_{D}) of about 0.1 to 10 nM. According to one embodiment, the humanized mAb or the fragment of the present invention binds STn glycan with an equilibrium dissociation constant (K_{D}) of about 0.02 to 2 nM. According to one embodiment, the humanized mAb or the fragment of the present invention binds STn glycan with an equilibrium dissociation constant (K_{D}) of about 0.02 to 1 nM. According to one embodiment, the humanized mAb or the fragment of the present invention binds STn glycan with an equilibrium dissociation constant (K_{D}) of about 0.02 to 0.5 nM.

According to some embodiments, the inhibitions constant (Ki) of the humanized mAb of the present invention or of the fragment thereof is from 30 to 500 nM, from 40 to 300 nM, from 50 to 200 nM or from 50 to 150 nM.

According to any one of the above embodiments, the selectivity (i.e. selectivity in cross reaction) of the humanized mAb or the fragment of the present invention to STn glycan is at least 90%. As used herein, the term “selectivity” for an antibody refers to an antibody that binds to a certain carbohydrate antigen but not too closely structurally related carbohydrates. The selectivity is identified as known in the art, e.g. as described in the Examples. According to another embodiment, the selectivity in cross reaction is at least 95% or at least 98%. According to one embodiment, the closely structurally related carbohydrate is Tn. According to one embodiment, the selectivity in cross reaction to STn glycan versus Tn glycan is at least 97% or at least 98%.

According to some embodiments, the humanized mAbs of fragments thereof have lower recognition by pooled human IgG antibodies than the original monoclonal antibodies, e.g. from 10 to 70% lowered binding of from 15 to 60% lower binding.

According to some embodiments, the humanized mAb or the fragment of the present invention binds STn glycan with an equilibrium dissociation constant (K_{D}) of about 0.01 to 100 nM and has selectivity to STn glycan in cross-reaction versus Tn glycan of at least 97% or at least 98%.

According to any one of the above embodiments, the heavy chain of the humanized mAb or the fragment of the present invention has a structure selected from the of IgG, IgA. IgD. IgE or IgM class (type). According to one embodiment, the mAb has an IgG structure. According to one embodiment, the heavy chain constant region is selected from the group consisting of: human IgG1, human IgG2, human, IgG3, human IgG4, mouse IgG1, mouse IgG2a, mouse IgG2b, mouse IgG3. According to other embodiments, the light chain constant region is selected from kappa and lambda.

According to some embodiments, the present invention provides a conjugate of the humanized mAb or of the fragment of the present invention. The term “conjugate” as used herein refers to the association of an antibody or a fragment thereof with another moiety. According to some embodiments, the moiety is a tag or label and the conjugate comprises a label. The term “tag” or “label” refers to a moiety which is attached, conjugated, linked or bound to, or associated with a compound such as the antibody or antibody fragment of the present invention and which may be used as a means of, for example, identifying, detecting and/or purifying the compound. Tags or labels include haemagglutinin tag, myc tag, poly-histidine tag, protein A, glutathione S transferase, Glu-Glu affinity tag, substance P, FLAG peptide, biotin and streptavidin binding peptide, enzyme, GFP, and rhodamine. According to some embodiments, the label is a fluorescent label.

The term “moiety” as used herein refers to a part of a molecule, which lacks one or more atom(s) compared to the corresponding molecule. The term “moiety”, as used herein, further relates to a part of a molecule that may include either whole functional groups or parts of functional groups as substructures.

According to some embodiments, the moiety is an active moiety. The term “active agent” and “active moiety” are used herein interchangeably and refer to an agent that has biological activity, pharmacologic effects and/or therapeutic utility.

According to some embodiments, the conjugate comprises the humanized mAb or fragment thereof and a tag. According to some embodiments, the conjugate comprises the humanized mAb or fragment thereof and an active moiety. According to some embodiments, the active moiety is an anti-cancer active moiety. According to some embodiments, the active moiety is an anti-cancer moiety. The term “anti-cancer”, “anti-neoplastic” and “anti-tumor” when referred to a compound, an agent or a moiety are used herein interchangeably and refer to a compound, drug, antagonist, inhibitor, or modulator such as immunomodulatory having anticancer properties or the ability to inhibit or prevent the growth, function or proliferation of and/or causing destruction of cells,” and in particular tumor cells. Therapeutic agents suitable in an anti-neoplastic composition for treating cancer include, but not limited to, chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, an anti-cancer agent is a chemotherapeutic. Thus, according to some embodiments, the present invention provides a conjugate of the humanized mAb of the present invention or of a fragment thereof and an anti-cancer moiety such as chemotherapeutic agents, radioactive isotopes, toxins, cytokines such as interferons, immunostimulating agents, immunomodulating agents and antagonistic agents targeting cytokines, cytokine receptors or antigens associated with tumor cells. According to another embodiment, the present invention provides a conjugate of the fragment of the mAb of the present invention and the anti-cancer moiety.

According to some embodiments, the present invention provides a conjugate of a humanized monoclonal antibody (mAb) or a fragment thereof that specifically binds to Sialyl Tn (STn) glycan wherein the VH domain comprises or consists of amino acid sequence SEQ ID NO: 28 and the VL domain comprises or consists of amino acid sequence SEQ ID NO: 29.

According to some embodiments, the present invention provides a conjugate of a humanized monoclonal antibody (mAb) or a fragment thereof that specifically binds to Sialyl Tn (STn) glycan wherein the VH domain comprises or consists of amino acid sequence SEQ ID NO: 28 and the VL domain comprises or consists of amino acid sequence SEQ ID NO: 30.

According to some embodiments, the present invention provides a conjugate of an scFv that specifically binds to Sialyl Tn (STn) glycan comprising or consisting of an amino acid sequence selected from SEQ ID NO: 31 and 32.

According to another aspect, the present invention provides a chimeric antigen receptor (CAR) comprising the humanized mAb or the fragment thereof of the present invention as described in any one of the above aspects and embodiments. All terms, embodiments and definitions defined in any one of the above aspects apply and are encompassed herein as well. According to some embodiments, the CAR comprises an antigen-binding domain comprising a heavy-chain variable domain (VH) and a light-chain variable domain (VL), wherein the VH and the VL each comprises three complementarity determining regions (CDRs) and four framework regions (FR). According to some embodiments, the CAR comprises an antigen-binding domain comprising a heavy-chain variable domain (VH) and a light-chain variable domain (VL), wherein the VH and the VL each comprises three complementarity determining regions (CDRs) and four framework regions (FR), wherein the VH comprises amino acid sequence SEQ ID NO:1 in which at least 9 amino acids in the framework domains are substituted and the VL comprises amino acid sequence SEQ ID NO:2 in which at least 9 amino acids in the framework domains are substituted. According to another embodiment, the CAR comprises an antigen-binding domain comprising a heavy-chain variable domain (VH) and a light-chain variable domain (VL). According to some embodiments, the CAR comprises a scFv of the present invention. According to some embodiments, the CAR comprises an scFv comprising an antigen-binding domain comprising a heavy-chain variable domain (VH) and a light-chain variable domain (VL), wherein the VH and the VL each comprises three complementarity determining regions (CDRs) and four framework regions (FR).

According to some embodiments, the CAR comprises an antigen-binding domain comprising that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO:1 in which from 9 to 16 amino acids in the framework regions are substituted and the VL domain comprises amino acid sequence SEQ ID NO:2 in which from 9 to 20 amino acids in the framework regions are substituted.

According to some embodiments, the CAR comprises an antigen-binding domain that specifically binds to Sialyl Tn (STn) glycan wherein the VH domain comprises or consists of amino acid sequence SEQ ID NO: 28 and the VL domain comprises or consists of amino acid sequence SEQ ID NO: 29.

According to some embodiments, the CAR comprises an antigen-binding domain that specifically binds to Sialyl Tn (STn) glycan wherein the VH domain comprises or consists of amino acid sequence SEQ ID NO: 28 and the VL domain comprises or consists of amino acid sequence SEQ ID NO: 30.

According to some embodiments, the CAR comprises an antigen-binding domain comprising an scFv that specifically binds to Sialyl Tn (STn) glycan comprising or consisting of amino acid sequence SEQ ID NO: 31. According to some embodiments, the CAR comprises an antigen-binding domain comprising an scFv that specifically binds to Sialyl Tn (STn) glycan comprising or consisting of amino acid sequence SEQ ID NO: 32.

The terms “chimeric antigen receptor” or “CAR” are used herein interchangeably and refer to engineered recombinant polypeptide or receptor which is grafted onto cells and comprises at least (1) an extracellular domain comprising an antigen-binding region, e.g., a single-chain variable fragment of an antibody or a whole antibody, (2) a transmembrane domain to anchor the CAR into a cell, and (3) one or more cytoplasmic signaling domains (also referred to herein as “an intracellular signaling domains”). The extracellular domain comprises an antigen-binding domain (ABD) and optionally a spacer or hinge region. The antigen-binding domain of the CAR targets a specific antigen. The targeting regions may comprise full length heavy chain, Fab fragments, or single chain variable fragment (scFv). The term “transmembrane domain” refers to the region of the CAR, which crosses or

bridges the plasma membrane. The transmembrane domain of the CAR of the invention is the transmembrane region of a transmembrane protein, an artificial hydrophobic sequence or a combination thereof. According to some embodiments, the term comprises also the transmembrane domain together with an extracellular spacer or hinge region.

The term “intracellular domain” refers to the intracellular part of the CAR and may be an intracellular domain of T cell receptor or of any other receptor (e.g., TNFR superfamily member) or portion thereof, such as an intracellular activation domain (e.g., an immunoreceptor tyrosine-based activation motif (ITAM)-containing T cell activating motif), an intracellular costimulatory domain, or both.

The CAR of the present invention comprises a transmembrane domain (TM domain), one or more costimulatory domains and an activation domain.

In one embodiment of the invention, the CAR includes a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154 or an analog thereof. According to one embodiment, the TM domain is a TM domain of a receptor selected from CD28 and CD8, or an analog thereof having at least 85% amino acid identity to the original sequence.

In some embodiments of the invention, the CAR comprises a costimulatory domain, e.g., a costimulatory domain comprising a functional signaling domain of a protein selected from the group consisting of OX40, CD2, CD27, CD28, CD5, ICAM-1, LFA-1 (CD11a/CD18), ICOS (CD278), 4-1BB (CD137), an analog thereof and a combination thereof. According to one embodiment, the costimulatory domain is selected from a costimulatory domain of a protein selected from CD28, 4-1BB, OX40, an analog thereof having at least 85% amino acid identity to the original sequence, and any combination thereof. According to some embodiments, the CAR of the present invention comprises two or more costimulatory domains. According to one embodiment, the CAR comprises costimulatory domains of CD28 and 4-1BB. According to some embodiments, the costimulatory domains of CD28 comprises amino acid sequence SEQ ID NO: 12.

According to one embodiment, the TM domain and the costimulatory domain of the CAR are both derived from CD28. According to one embodiment, the TM domain and the costimulatory domain of the CAR comprise amino acid sequence SEQ ID NO: 13. According to one embodiment, the TM domain and the costimulatory domain of the CAR comprise an analog of amino acid sequence SEQ ID NO: 13 or having at least 85% amino acid identity.

According to some embodiments, the antigen-binding domain is linked to the TM domain via a spacer.

According to any one of the above embodiments, the CAR comprises an activation domain selected from FcRγ (gamma) and CD3-ζ (CD3-zetta) activation domains, or any other sequence that contains an intracellular tyrosine activating motif (ITAM). According to one embodiment, the activation domain is FcRγ domain. The terms “activation domain” and “signaling domain” may be used interchangeably. According to one embodiment, the activation domain comprises amino acid sequence SEQ ID NO: 14. According to one embodiment, the activation domain comprises an analog of amino acid sequence SEQ ID NO: 14, having at least 85% amino acid identity to it.

According to some embodiments, the CAR of the present invention comprises an scFv according to any one of the above embodiments, a TM domain of a receptor selected from CD28 and CD8, a costimulatory domain selected from the domain of CD28, 4-1BB, OX40 and a combination thereof, and an activation domain is selected from FcRγ and CD3-activation domains. According to some embodiments, the CAR of the present invention comprises an scFv of the present invention, a TM domain of a receptor selected from CD28 and CD8, a costimulatory domain selected from the domain of CD28, 4-1BB, OX40 and a combination thereof, and an activation domain is selected from FcRγ and CD3-ζ activation domains.

The term “CD28” refers to cluster of differentiation 28 protein. In some embodiments, the CD28 is a human CD28.

The term “CD8” refers to cluster of differentiation 8 protein being a transmembrane glycoprotein and serving as a co-receptor for the T cell receptor. According to one embodiment, the CD8 is a human CD8.

The terms “ICOS” and “Inducible T-cell COStimulator” refer to CD278 which is a CD28-superfamily costimulatory molecule. According to one embodiment, the ICOS is a human ICOS.

The term “4-1BB” refers to a CD137 protein which is a member of the tumor necrosis factor receptor family and has costimulatory activity for activated T cells. According to one embodiment, 4-1BB is a human 4-1BB.

The terms “CD3ζ” and “CD3-zetta” refer to a ζ (zetta) chain of CD3 (cluster of differentiation 3) T cell co-receptor participating in activation of both the cytotoxic and helper T cells. According to one embodiment, CD3ζ comprises an immunoreceptor tyrosine-based activation motif (ITAM). According to one embodiment, the CD3ζ is human CD3ζ. CD3ζ is sometimes also referred as CD247.

The term “FcRγ” refers to Fc gamma receptors, which generate signals within their cells through ITAM. These are immunoglobulin superfamily receptors that are found on various innate as well as adoptive immune cells, where the extracellular part binds IgGs the activation signal is transduced through two ITAM located on its cytoplasmic tail.

According to any one of the above embodiments, the CAR further comprises a leading peptide. According to one embodiment, the leading peptide is located N-terminally to the ABD.

The term “leader peptide”, “leading peptide”, “lead peptide”, “signaling peptide” and “signal peptide” are used herein interchangeable and refer to a peptide that translocates or prompts translocation of the target protein to cellular membrane. According to one embodiment, the leading peptide is located N-terminally to the ABD. According to one embodiment, the leading peptide has amino acid sequence SEQ ID NO: 15 or an analog thereof having at least 85% amino acid identity.

According to one embodiment, the TM domain and the costimulatory domain of the CAR are both derived from CD28. According to one embodiment, the TM domain and the costimulatory domain have amino acid sequence SEQ ID NO: 13. According to another embodiment, the TM domain and the costimulatory domain have an amino acid sequence which is an analog of SEQ ID NO: 13 having at least 85% amino acid identity to SEQ ID NO: 13. According to some embodiments, the antigen-binding domain is linked to the TM domain via a spacer. According to one embodiment, the spacer comprises amino acid sequence comprising from 1 to 6 repetitions, such as 1, 2, 3, 4, 5 or 6 repetitions, of amino acid sequence SEQ ID NO: 9. According to one embodiment, the spacer comprises an amino acid sequence comprising 2 repetitions of amino acid sequence SEQ ID NO: 9. According to another embodiment, the spacer comprises amino acid SEQ ID NO: 11. According to any one of the above embodiments, the sequences of the TM domain, a costimulatory domain, an activation domain and a leading peptide are as set forth in amino acid sequences SEQ ID NOs: 13, 14 and 15, respectively or an analog thereof having at least 85% amino acid identity.

According to some embodiment, the present invention provides a CAR that specifically binds to Sialyl Tn (STn) glycan and comprises amino acid sequence SEQ ID NO: 41. According to some embodiment, the present invention provides a CAR that specifically binds to Sialyl Tn (STn) glycan and consists of amino acid sequence SEQ ID NO: 41. According to some embodiment, the present invention provides a CAR that specifically binds to Sialyl Tn (STn) glycan and comprises amino acid sequence SEQ ID NO: 42. According to some embodiment, the present invention provides a CAR that specifically binds to Sialyl Tn (STn) glycan and consists of amino acid sequence SEQ ID NO: 42.

According to some embodiments, the CAR of the present invention may further comprise a tag sequence. The term “tag” or “label” refers to a moiety which is attached, conjugated, linked or bound to, or associated with, a compound (for example a protein, peptide, amino acid, nucleic acid and/or carbohydrate) and which may be used as a means of, for example, identifying, detecting and/or purifying a compound. According to some embodiments, the tag is selected haemagglutinin tag, myc tag, poly-histidine tag, protein A, glutathione S transferase, Glu-Glu affinity tag, substance P. FLAG peptide, streptavidin (strep) binding peptide and human FC tag. According to some embodiments, the tag is a strep-tag, e.g., comprising an amino acid sequence SEQ ID NO: 46 and/or encoded by a nucleic acid sequence SEQ ID NO: 47. According to some embodiments, the tag is located at the C-terminus of the scFv.

According to some embodiments, the scFv or scFv comprising a tag are spaced from the TM domain by a hinge or a spacer, e.g. comprising from 1 to 4 repetitions of amino acid sequence SEQ ID NO: 9.

According to some embodiment, the present invention provides a CAR that specifically binds to Sialyl Tn (STn) glycan and comprises or consists of amino acid sequence SEQ ID NO: 48. According to some embodiment, the present invention provides a CAR that specifically binds to Sialyl Tn (STn) glycan and comprises or consists of amino acid sequence SEQ ID NO: 49. [with strep-tag]

According to another aspect, the present invention provides a nucleic acid molecule encoding at least one chain of the humanized monoclonal antibody or fragment thereof as described in any one of the above embodiments and aspects or the CAR of the present invention. All terms, embodiments and definitions defined in any one of the above aspects apply and are encompassed herein as well. According to some embodiments, the nucleic acid molecule encodes at least one chain of the humanized monoclonal antibody or fragment thereof. According to some embodiments, the nucleic acid molecule encodes the CAR of the present invention.

The term “nucleic acid molecule” refers to a single-stranded or double-stranded sequence (polymer) of deoxyribonucleotides or ribonucleotides. The terms “nucleic acid” and “polynucleotide” are used herein interchangeably. According to some embodiments, the nucleic acid molecule is an isolated nucleic acid molecule. The term “isolated nucleic acid” as used herein denotes that the nucleic acid is essentially free of other cellular components with which it is associated in the cell. It can be, for example, a homogeneous state and may be dry or in the state of a solution, such as an aqueous solution. Purity and homogeneity are typically determined using analytical chemistry techniques such as polyacrylamide gel electrophoresis or high-performance liquid chromatography. The term “encoding” refers to the ability of a nucleotide sequence to code for one or more amino acids. The term does not require a start or stop codon. An amino acid sequence can be encoded in any one of six different reading frames provided by a polynucleotide sequence and its complement.

According to some embodiments, the nucleic acid molecule encodes a VH domain of an antigen-binding domain that specifically binds to Sialyl Tn (STn) glycan, wherein the VH domain comprises an amino acid sequence SEQ ID NO:1 in which from 9 to 16 amino acids in the framework regions. According to some embodiments, the nucleic acid molecule encodes a VL domain of an antigen-binding domain comprising that specifically binds to Sialyl Tn (STn) glycan, wherein the VL domain comprises amino acid sequence SEQ ID NO:2 in which from 9 to 20 amino acids in the framework regions are substituted.

According to some embodiments, the nucleic acid molecule encodes the VH domain comprising or consisting of amino acid sequence SEQ ID NO: 28. According to some embodiments, the nucleic acid molecule encodes the VL domain comprising or consisting of amino acid sequence SEQ ID NO: 29. According to some embodiments, the nucleic acid molecule encodes the VH domain comprising or consisting of amino acid sequence SEQ ID NO: 28 and the VL domain comprising or consisting of amino acid sequence SEQ ID NO: 29.

According to some embodiments, the nucleic acid molecule encodes the VL domain comprising or consisting of amino acid sequence SEQ ID NO: 30. According to some embodiments, the nucleic acid molecule encodes the VH domain comprising or consisting of amino acid sequence SEQ ID NO: 28 and the VL domain comprising or consisting of amino acid sequence SEQ ID NO: 30.

According to some embodiments, the nucleic acid molecule encodes an scFv that specifically binds to Sialyl Tn (STn) glycan comprising or consisting of amino acid sequence SEQ ID NO: 31. According to some embodiments, the nucleic acid molecule encodes an scFv that specifically binds to Sialyl Tn (STn) glycan comprising or consisting of amino acid sequence SEQ ID NO: 32.

According to some embodiments, the nucleic acid molecule encodes further the sequences of the TM domain, a costimulatory domain, an activation domain and a leading peptide as set forth in amino acid sequences SEQ ID NOs: 13, 14 and 15, respectively or an analog thereof having at least 85% amino acid identity.

According to some embodiments, the nucleic acid molecule encodes a CAR that specifically binds to Sialyl Tn (STn) glycan and comprising amino acid sequence SEQ ID NO: 41. According to some embodiments, the nucleic acid molecule encodes a CAR that specifically binds to Sialyl Tn (STn) glycan and consisting of amino acid sequence SEQ ID NO: 41. According to some embodiments, the nucleic acid molecule encodes a CAR that specifically binds to Sialyl Tn (STn) glycan and comprising amino acid sequence SEQ ID NO: 42. According to some embodiments, the nucleic acid molecule encodes a CAR that specifically binds to Sialyl Tn (STn) glycan and consisting of amino acid sequence SEQ ID NO: 42. According to some embodiments, the nucleic acid molecule encodes a CAR that specifically binds to Sialyl Tn (STn) glycan and comprising or consisting of amino acid sequence SEQ ID NO: 48. According to some embodiments, the nucleic acid molecule encodes a CAR that specifically binds to Sialyl Tn (STn) glycan and comprising or consisting of amino acid sequence SEQ ID NO: 49.

According to some embodiments, the nucleic molecule comprises a nucleic acid sequence SEQ ID NO: 33. According to some embodiments, the nucleic molecule comprises a nucleic acid sequence SEQ ID NO: 45. According to some embodiments, the nucleic molecule comprises a nucleic acid sequence SEQ ID NO: 34. According to some embodiments, the nucleic molecule comprises a nucleic acid sequence SEQ ID NO: 35.

According to some embodiments, the nucleic molecule comprises a nucleic acid sequences SEQ ID NO: 45 and 34. According to some embodiments, the nucleic molecule comprises a nucleic acid sequences SEQ ID NO: 33 and 35.

According to some embodiments, the nucleic molecule comprises a nucleic acid sequence SEQ ID NO: 36. According to some embodiments, the nucleic molecule comprises a nucleic acid sequence SEQ ID NO: 37. According to some embodiments, the nucleic molecule further comprises one or more a nucleic acid sequences selected from SEQ ID NO: 22, 23, and 24.

According to some embodiments, the nucleic molecule comprises a nucleic acid sequence SEQ ID NO: 43. According to some embodiments, the nucleic molecule comprises a nucleic acid sequence SEQ ID NO: 44. According to some embodiments, the nucleic molecule comprises a nucleic acid sequence SEQ ID NO: 50. According to some embodiments, the nucleic molecule comprises a nucleic acid sequence SEQ ID NO: 51.

The terms “homolog” “variant”, “DNA variant”, “sequence variant” and “polynucleotide variant” are used herein interchangeably and refer to a DNA polynucleotide having at least 70% sequence identity to the parent polynucleotide. The variant may include mutations such as deletion, addition or substitution such that the mutations do not change the open reading frame and the polynucleotide encodes a peptide or a protein having substantially similar structure and function as a peptide or a protein encoded by the parent polynucleotide. Thus, according the variant encodes to a polypeptide or protein that have the same function as the protein polypeptide or protein encoded by the original polynucleotide. According to some embodiments, the variants are conservative variants. The term “conservative variants” as used herein refers to variants in which a change of one or more nucleotides in a given codon position results in no alteration in the amino acid encoded at that position. Thus, the peptide or the protein encoded by the conservative variants has 100% sequence identity to the peptide or the protein encoded by the parent polynucleotide. According to some embodiments, the variant is a non-conservative variant encoding to a peptide or a protein being a conservative analog of the peptide of the protein encoded by the parent polynucleotide. According to some embodiments, the variant has at least 75%, at least 80% at least 85%, at least 90%, at least 95%, at least 98% or at least 99% sequence identity to the original nucleic acid sequence. According to one embodiment, the variant is a conservative variant.

According to another aspect, the present invention provides a nucleic acid construct comprising the nucleic acid of the present invention, operably linked to a promoter. All terms, embodiments and definitions defined in any one of the above aspects apply and are encompassed herein as well.

The terms “operably linked”, “operatively linked”, “operably encodes”, “operably bound” and “operably associated” are used herein interchangeably and refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

According to some embodiments, the nucleic acid construct comprises a nucleic acid sequence SEQ ID NO: 33. According to some embodiments, the nucleic acid construct comprises a nucleic acid sequence SEQ ID NO: 34. According to some embodiments, the nucleic acid construct comprises a nucleic acid sequence SEQ ID NO: 35. According to some embodiments, the nucleic acid construct comprises a nucleic acid sequence SEQ ID NO: 45. According to some embodiments, the nucleic acid construct comprises nucleic acid sequences SEQ ID NO: 45 and 34. According to some embodiments, the nucleic acid construct comprises nucleic acid sequences SEQ ID NO: 33 and 35. According to some embodiments, the nucleic acid construct comprises a nucleic acid sequence SEQ ID NO: 36 According to some embodiments, the nucleic acid construct comprises a nucleic acid sequence SEQ ID NO: 37. According to some embodiments, the nucleic acid construct further comprises one or more a nucleic acid sequences selected from SEQ ID NO: 22, 23, and 24. According to some embodiments, the nucleic acid construct comprises a nucleic acid sequence SEQ ID NO: 43. According to some embodiments, the nucleic acid construct comprises a nucleic acid sequence SEQ ID NO: 44. According to some embodiments, the nucleic acid construct comprises a nucleic acid sequence SEQ ID NO: 50. According to some embodiments, the nucleic acid construct comprises a nucleic acid sequence SEQ ID NO: 51.

According to another aspect, the present invention provides a vector comprising the nucleic acid molecule or nucleic acid construct of the present invention. All terms, embodiments and definitions defined in any one of the above aspects apply and are encompassed herein as well.

The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, sequence which is able to transform host cells and optionally capable of replicating in a host cell. The vector may be integrated into the cellular genome or may exist extra-chromosomally (e.g., autonomous replicating plasmid with an origin of replication). The vector may contain an optional marker suitable for use in the identification of transformed cells, e.g., tetracycline resistance or ampicillin resistance. A cloning vector may or may not possess the features necessary for it to operate as an expression vector. Any vector known in the art is envisioned for use in the practice of this invention. According to other embodiments, the vector is a virus, e.g. a modified or engineered virus. The modification of a vector may include mutations, such as deletion or insertion mutation, gene deletion or gene inclusion. In particular, a mutation may be done in one or more regions of the viral genome. Such mutations may be introduced in a region related to internal structural proteins, replication, or reverse transcription function. Other examples of vector modification are deletion of certain genes constituting the native infectious vector such as genes related to the virus' pathogenicity and/or to its ability to replicate. Any virus can be attenuated by the methods disclosed herein. According to some embodiments, the vector is a virus selected from lentivirus, adenovirus, modified adenovirus and retrovirus. In one particular embodiment, the vector is lentivirus. According to other embodiments, the vector is a plasmid.

According to another aspect, the present invention provides a cell comprising the humanized monoclonal antibody or the antibody fragment thereof, the CAR, the nucleic acid molecule, the nucleic acid construct or the vector of the present invention. All terms, embodiments and definitions defined in any one of the above aspects apply and are encompassed herein as well. According to some embodiments, the cell comprises the humanized monoclonal antibody. According to one embodiment, the cell comprises a fragment of the humanized monoclonal antibody of the present invention. According to other embodiments, the cell comprises, expresses or is capable of expressing the CAR or the present invention. According to yet another embodiment, the cell comprises the nucleic acid molecule, the nucleic acid construct or the vector of the present invention encoding the humanized monoclonal antibody or the antibody fragment thereof or the CAR of the present invention.

According to some embodiments, the cell comprises a humanized monoclonal antibody (mAb) that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO:1 in which from 9 to 16 amino acids in the framework regions are substituted and the VL domain comprises amino acid sequence SEQ ID NO:2 in which from 9 to 20 amino acids in the framework regions are substituted. According to some embodiments, the present invention provides a fragment of the humanized monoclonal antibody (mAb) that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO: 1 in which from 9 to 16 amino acids in the framework regions are substituted and the VL domain comprises amino acid sequence SEQ ID NO:2 in which from 9 to 20 amino acids in the framework regions are substituted.

According to some embodiments, the present invention provides a cell comprising a humanized monoclonal antibody (mAb) or a fragment thereof that specifically binds to Sialyl Tn (STn) glycan wherein the VH domain comprises the amino acid sequence SEQ ID NO: 28 the VL domain comprises the amino acid sequence SEQ ID NO: 29. According to some embodiments, the present invention provides a cell comprising a humanized monoclonal antibody (mAb) or a fragment thereof that specifically binds to Sialyl Tn (STn) glycan wherein the VH domain consists of amino acid sequence SEQ ID NO: 28 and the VL domain consists of the amino acid sequence SEQ ID NO: 29.

According to some embodiments, the present invention provides a cell comprising a humanized monoclonal antibody (mAb) or a fragment thereof that specifically binds to Sialyl Tn (STn) glycan wherein the VH domain comprises the amino acid sequence SEQ ID NO: 28 the VL domain comprises the amino acid sequence SEQ ID NO: 30. According to some embodiments, the present invention provides a cell comprising a humanized monoclonal antibody (mAb) or a fragment thereof that specifically binds to Sialyl Tn (STn) glycan wherein the VH domain consists of the amino acid sequence SEQ ID NO: 28 the VL domain consists of the amino acid sequence SEQ ID NO: 30.

According to some embodiments, the present invention provides a cell comprising an scFv that specifically binds to Sialyl Tn (STn) glycan wherein the VH domain comprises the amino acid sequence SEQ ID NO: 28 the VL domain comprises the amino acid sequence SEQ ID NO: 29. According to some embodiments, the VH domain consists of amino acid sequence SEQ ID NO: 28 and the VL domain consists of the amino acid sequence SEQ ID NO: 29.

According to some embodiments, the present invention provides a cell comprising an scFv that specifically binds to Sialyl Tn (STn) glycan wherein the VH domain comprises the amino acid sequence SEQ ID NO: 28 the VL domain comprises the amino acid sequence SEQ ID NO: 30. According to some embodiments, the VH domain consists of amino acid sequence SEQ ID NO: 28 and the VL domain consists of the amino acid sequence SEQ ID NO: 30.

According to some embodiments, the cell comprises a scFv comprising amino acid sequence SEQ ID NO: 31.

According to some embodiments, provides a cell comprises ab scFv comprising amino acid sequence SEQ ID NO: 32.

According to some embodiments, the present invention provides a cell comprising the CAR of the present invention. According to some embodiments, the CAR comprises an antigen-binding domain that specifically binds to Sialyl Tn (STn) glycan wherein the VH domain comprises or consists of amino acid sequence SEQ ID NO: 28 and the VL domain comprises or consists of an amino acid sequence selected from SEQ ID NO: 29 and 30. According to some embodiments, the cell comprising a CAR comprising an antigen-binding domain comprising an scFv that specifically binds to Sialyl Tn (STn) glycan comprising or consisting of amino acid sequence SEQ ID NO: 31. According to some embodiments, the CAR comprises an antigen-binding domain comprising an scFv that specifically binds to Sialyl Tn (STn) glycan comprising or consisting of amino acid sequence SEQ ID NO: 32. According to some embodiment, the present invention provides a cell comprising a CAR that specifically binds to Sialyl Tn (STn) glycan and comprises amino acid sequence SEQ ID NO: 41. According to some embodiment, the present invention provides a cell comprising a CAR that specifically binds to Sialyl Tn (STn) glycan and comprises of amino acid sequence SEQ ID NO: 41. According to some embodiment, the present invention provides a cell comprising a CAR that specifically binds to Sialyl Tn (STn) glycan and comprises amino acid sequence SEQ ID NO: 42. According to some embodiment, the present invention provides a cell comprising a CAR that specifically binds to Sialyl Tn (STn) glycan and comprises amino acid sequence SEQ ID NO: 48. According to some embodiment, the present invention provides a cell comprising a CAR that specifically binds to Sialyl Tn (STn) glycan and comprises amino acid sequence SEQ ID NO: 49. According to some embodiments, the present invention provides a cell comprising a nucleic acid molecule encoding said monoclonal antibodies, fragments or CARs. According to some embodiments, the cell comprises one or more nucleic molecules, constructs or vectors of the present invention comprising one or more nucleic acid sequences selected from SEQ ID NO: 33, 34, 35, 45, 36, 37, 43, 44, 50, and 51. According to some embodiments, the cells a capable or engineered to express the mAbs, fragments or CAR of the present invention.

According to some embodiments, the cell is selected from a bacterial, fungi, such as yeast, and mammalian cell. According to some embodiments, the cell is a mammalian cell. According to some embodiments, the cells are capable of producing or expressing or that produces or expresses the humanized monoclonal antibody or the antibody fragment of the present invention. According to one embodiment, the cell is a Hybridoma cell. According to another embodiment, the cell is a human cell. According to some embodiments, the cell is a leukocyte. According to some embodiments, the cell is selected from T cell and a natural killer (NK) cell. According to some embodiments, the present invention provides a T-cell genetically modified to express the CAR of the present invention.

According to some embodiment, the cells are T cells. Thus, according to some embodiments, the present invention provides T-cells comprising the CAR of the present invention. According to some embodiments, the T-cells comprise a CAR comprising the humanized mAb or the fragment thereof as described in any one of the above aspects and embodiments.

The term “T cell” refers to a type of white blood cell that can be distinguished from other white blood cells by the presence of a T cell receptor on the cell surface. There are several subsets of T cells, including, but not limited to, T helper cells (a.k.a. Tx cells or CD4⁺ T cells) and subtypes, including T_{H}1, T_{H}2, T_{H}3, T_{H}17, T_{H}9, and THE cells, cytotoxic T cells (i.e., Tc cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. Treg cells or suppressor T cells) and subtypes, including CD4⁺FOXP3⁺ T_{reg} cells, CD4⁺FOXP3⁻ T_{reg} cells, Tr1 cells, Th3 cells, and T_{reg} 17 cells, natural killer T cells (a.k.a. NKT cells), mucosal associated invariant T cells (MAITs), and gamma delta T cells (γδ T cells), including Vy9/Vδ2 T cells. Any one or more of the aforementioned or unmentioned T cells may be the target cell type for a method of use of the invention. According to some embodiments, the cells are T cells. According to some embodiments, the T-cells are selected from memory, regulatory, helper or natural killer T-cells. According to some embodiments, the T cell is selected are from CD4+ T-cell and a CD8+ T-cell. According to some embodiments, the T cell are CD4+ T-cell and a CD8+ T-cell. According to some embodiments, the cells are NK cells. According to some embodiments, the cells are NK T-cells.

According to any one of the above embodiments, the humanized mAb of the present invention or the functional fragment thereof is capable of activating T cells. According to one embodiment, the mAb of the present invention of the functional fragment thereof is capable of promoting T cells proliferation, generation and/or survival. According to some embodiments, the T-cells are selected from memory, regulatory, helper and natural killer T-cells. As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. Activation results in clonal expansion of T cells, upregulation of activation markers on the cell surface, differentiation into effector cells, induction of cytotoxicity or cytokine secretion, induction of apoptosis, or a combination thereof. As used herein, “improving cell survival” and “promoting cell survival” refers to an increase in the number of cells that survive a given condition or period, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition.

According to some embodiments, the present invention provides a plurality of T cells comprising the CAR of the present invention. According to some embodiments, the CAR comprises an antigen-binding domain comprising that specifically binds to Sialyl Tn (STn) glycan wherein the VH domain comprises or consists of amino acid sequence SEQ ID NO: 28 and the VL domain comprises or consists of an amino acid sequence selected from SEQ ID NO: 29 and 30. According to some embodiments, the T cells comprise a CAR comprising as an antigen-binding domain an scFv that specifically binds to Sialyl Tn (STn) glycan and comprising or consisting of amino acid sequence SEQ ID NO: 31. According to some embodiments, the CAR comprises as an antigen-binding domain an scFv that specifically binds to Sialyl Tn (STn) glycan and comprising or consisting of amino acid sequence SEQ ID NO: 32. According to some embodiment, the T cells comprise a CAR that specifically binds to Sialyl Tn (STn) glycan and comprise amino acid sequence SEQ ID NO: 41. According to some embodiment, the T cells comprise a CAR that specifically binds to Sialyl Tn (STn) glycan and consists of amino acid sequence SEQ ID NO: 41. According to some embodiment, the T cells comprise a CAR that specifically binds to Sialyl Tn (STn) glycan and comprises amino acid sequence SEQ ID NO: 42. According to some embodiment, the T cells comprise a CAR that specifically binds to Sialyl Tn (STn) glycan and consists of amino acid sequence SEQ ID NO: 42. According to some embodiment, the T cells comprise a CAR that specifically binds to Sialyl Tn (STn) glycan and comprises or consists of amino acid sequence SEQ ID NO: 48. According to some embodiment, the T cells comprise a CAR that specifically binds to Sialyl Tn (STn) glycan and comprises or consists of amino acid sequence SEQ ID NO: 49. According to some embodiments, the T cells comprise a nucleic acid molecule encoding said monoclonal antibodies, fragments or CARs. According to some embodiments, the T cells comprise one or more nucleic molecules, constructs or vectors comprising one or more nucleic acid sequences selected from SEQ ID NO: 33, 34, 35, 45 36, 37, 43, 44, 50 and 51. According to some embodiments, the T cells are capable or engineered to express the CAR of the present invention. According to some embodiments, the T cells are selected are from CD4+ T-cell and a CD8+ T-cell. According to some embodiments, the T cells are a combination of CD4+ T-cell and a CD8+ T-cell. According to some embodiments, the cells are NK cells. According to some embodiments, the cells are NK T− cells.

According to another aspect, the present invention provides a composition comprising a plurality of humanized monoclonal antibodies or antibody fragments, conjugates or cells of the present invention, and a carrier. All terms, embodiments and definitions defined in any one of the above aspects apply and are encompassed herein as well. The term “carrier” includes as a class any compound, solvent or composition useful in facilitating storage, stability, and use of the mAbs or fragments of the present invention.

According to some embodiments, the composition is a diagnostic composition. As used herein, the term “diagnostic” is meant to encompass both determining the susceptibility of one object to a particular disease or disease, determining whether one object currently has a particular disease or disease (e.g., identifying diabetes or complications thereof), determining a prognosis of one object hung on a particular disease or discase, or monitoring the state of the object to provide information about therapeutic efficacy of a particular drug.

According to some embodiments, the composition is a pharmaceutical composition and the carrier is a pharmaceutically acceptable carrier. Thus, according to some embodiments, the present invention provides a pharmaceutical composition comprising the humanized monoclonal antibody of the present invention, and a pharmaceutically acceptable carrier. According to one embodiment, the pharmaceutical composition comprises a plurality of the antibody fragments of the present invention, and a pharmaceutically acceptable carrier. According to some embodiment, the pharmaceutical composition comprises a plurality of conjugates of the present invention comprising the humanized monoclonal antibodies or fragments thereof of the present invention, and a pharmaceutically acceptable carrier. According to another embodiment, the pharmaceutical composition comprises a plurality of cells of the present invention, and a pharmaceutically acceptable carrier. According to another embodiment, the pharmaceutical composition comprises a plurality of T-cells comprising the CAR of the present invention, and a pharmaceutically acceptable carrier.

According to some embodiments, the pharmaceutical composition comprises a plurality of T cells comprising the CAR of the present invention. According to some embodiments, the CAR comprises an antigen-binding domain comprising that specifically binds to Sialyl Tn (STn) glycan wherein the VH domain comprises or consists of amino acid sequence SEQ ID NO: 28 and the VL domain comprises or consists of an amino acid sequence selected from SEQ ID NO: 29 and 30. According to some embodiments, the T cells comprise a CAR comprising an antigen-binding domain comprising a scFv that specifically binds to Sialyl Tn (STn) glycan comprising or consisting of amino acid sequence SEQ ID NO: 31. According to some embodiments, the CAR comprises an antigen-binding domain comprising a scFv that specifically binds to Sialyl Tn (STn) glycan comprising or consisting of amino acid sequence SEQ ID NO: 32. According to some embodiment, the T cells comprising a CAR that specifically binds to Sialyl Tn (STn) glycan and comprises or consists of an amino acid sequence selected from SEQ ID NO: 41. According to some embodiment, the T cells comprise a CAR that specifically binds to Sialyl Tn (STn) glycan and consisting of amino acid sequence SEQ ID NO: 42. According to some embodiment, the T cells comprise a CAR that specifically binds to Sialyl Tn (STn) glycan and consisting of amino acid sequence SEQ ID NO: 48. According to some embodiment, the T cells comprise a CAR that specifically binds to Sialyl Tn (STn) glycan and consisting of amino acid sequence SEQ ID NO: 49. According to some embodiments, the T cells comprise a nucleic acid molecule encoding said monoclonal antibodies, fragments or CARs. According to some embodiments, the T cells comprise one or more nucleic molecules, constructs or vectors comprising one or more nucleic acid sequences selected from SEQ ID NO: 33, 34, 35, 45, 36, 37, 43, 44, 50 and 51. According to some embodiments, the cells a capable or engineered to express the CAR of the present invention.

According to some embodiments, the present invention provides a pharmaceutical composition comprising a conjugate of the humanized monoclonal antibodies of the present invention. According to some embodiments, the conjugate is a conjugate of the mAb comprising an antigen-binding domain that specifically binds to Sialyl Tn (STn) glycan wherein the VH domain comprises or consists of amino acid sequence SEQ ID NO: 28 and the VL domain comprises or consists of an amino acid sequence selected from SEQ ID NO: 29 and 30. According to some embodiments, the antigen-binding domain comprises a scFv that specifically binds to Sialyl Tn (STn) glycan comprising or consisting of amino acid sequence SEQ ID NO: 31. According to some embodiments, the scFv that specifically binds to Sialyl Tn (STn) glycan comprises or consists of amino acid sequence SEQ ID NO: 32.

The term “pharmaceutical composition” as used herein refers to a composition comprising at least one active agent as disclosed herein, e.g., mAbs or fragments thereof, conjugates, CAR T-cells, formulated together with one or more pharmaceutically acceptable carriers.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The pharmaceutical compositions of the present invention may be prepared by conventional techniques, e.g., as described in Remington: The Science and Practice of Pharmacy, 19th Ed., 1995. The compositions may be in solid, semisolid or liquid form and may further include pharmaceutically acceptable fillers, carriers or diluents, and other inert ingredients and excipients. The compositions can be administered by any suitable route, e.g., orally, intravenously, parenterally, rectally or transdermally, the oral route being preferred. The dosage will depend on the state of the patient, and will be determined as deemed appropriate by the practitioner.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions, solid carriers or excipients such as, for example, lactose, starch or talcum or liquid carriers such as, for example, water, fatty oils or liquid paraffins.

Solutions or suspensions used for parenteral, intradermal, or subcutaneous application typically include the following components: a sterile diluent such as water for injection, saline solution, fixed oils, polyethylene glycols, glycerin, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

According to some embodiments, the pharmaceutical composition is formulated for a parenteral administration. According to one embodiment, the composition is formulated for subcutaneous, intraperitoneal (IP), IM, IV or intratumor administration. According to other embodiments, the pharmaceutical composition is formulated as a solution such as a sterile solution for injection.

According to any one of the above embodiments, the pharmaceutical composition of the present invention is for use in treating cancer. According to some embodiments, the pharmaceutical composition comprising the humanized mAb, fragments thereof or conjugates thereof is for use in treating cancer. According to some embodiments, the pharmaceutical composition comprising T-cells comprising the CAR of the present invention is for use in treating cancer. According to some embodiments, the cancer is a cancer overexpressing STn glycan. According to some embodiments, the cancer is selected from carcinoma and lymphoma. According to some embodiments, the cancer is selected from endometrial carcinoma, ovarian carcinoma, prostate adenocarcinoma, seminoma, diffuse type gastric adenocarcinoma, pancreatic and colon adenocarcinomas, lung adenocarcinoma and mantle cell lymphoma. According to one embodiment, the cancer is selected from hematological, breast, ovarian, pancreatic, colorectal, stomach, head and neck, liver, lung, oropharyngeal cancer, acute myeloid leukemia (AML) squamous cell carcinoma, melanoma and gallbladder cancer. According to one embodiment, the cancer is a breast cancer. According to some embodiment, the cancer is a Her-2 negative breast carcinoma. According to another embodiment, the cancer is an ovarian cancer. According to a further embodiment, the cancer is a colon cancer. According to one embodiment, the cancer is colon adenocarcinoma. According to one embodiment, the cancer is a colorectal cancer. According to another embodiment, the cancer is a stomach cancer. According to one embodiment, the cancer is a pancreatic cancer. According to one embodiment, the cancer is carcinoma. According to one embodiment, the cancer is a hematological cancer overexpressing STn glycan. According to another embodiment, the cancer is a pancreatic adenocarcinoma. According to yet another embodiment, the cancer is lung cancer. According to one embodiment, the cancer is lung adenocarcinoma. According to some embodiments, the cancer is squamous cell carcinoma. According to another embodiment, the cancer is pharynx squamous cell carcinoma.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

The use comprises administering the pharmaceutical composition of the present invention to the subject. According to any one of the above embodiments, the composition of the present invention is administered as known in the art. According to one embodiment, the composition is parenterally administered, e.g. IP. IV, IM, SC or intratumorally. According to some embodiments, the pharmaceutical composition of the present invention is administered via infusion, such as IV infusion. According to some embodiments, the composition is systemically administered. According to other embodiments, the composition is locally administered.

The terms “administering” or “administration of” a substance, a compound, the composition or an agent to a subject are used herein interchangeably and refer to an administration mode that can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both a direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition is parenterally administered. The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

According to some embodiments, the pharmaceutical composition of the present invention is co-administered with other anti-tumor therapy including but not limited to anticancer drugs, radiotherapy, immunotherapy and surgery. According to some embodiments, the therapeutic agents suitable for co-administration with the pharmaceutical composition of the present invention are selected from chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, an anti-cancer agent is a chemotherapeutic.

According to another aspect, the present invention provides a method for treating cancer in a subject in need thereof comprising administering a therapeutically effective amount of the humanized mAb antibodies or functional fragments thereof of the present invention. According to another embodiment, the method comprises administering a pharmaceutical composition comprising the humanized mAb or fragments thereof to the subject. According to another embodiment, the method comprises administering CAR of the present invention to the subject. According to one embodiment, the method comprises administering T-cells comprising the CAR of the present invention to the subject. According to yet another embodiment, the method comprises administering a pharmaceutical composition comprising cells or expressing the humanized mAb or the fragments thereof to the subject. According to some embodiments, the humanized mAb antibodies or functional fragments thereof are formulated with a delivery system such as liposomes.

According to yet another aspect, the present invention provides a use of the humanized mAb antibodies or functional fragments thereof, the CARSm the conjugates or the cells, such as T-cells, of the present invention for preparing a medicament for treating cancer.

The present invention further provides in another aspect a method of detecting, determining, and/or quantifying the presence of STn glycan in a biological sample, the method comprises contacting the biological sample with the monoclonal antibodies, antibody fragments or conjugates of the present invention and subsequently detecting, determining, and/or quantifying the presence or the amount of STn glycan in the sample. All terms, embodiments and definitions defined in any one of the above aspects apply and are encompassed herein as well. These methods allow detecting, determining, and/or quantifying the expression STn on cells by contacting the biological sample comprising the cells with the monoclonal antibodies, antibody fragments or conjugates of the present invention. According to some embodiments, detecting, determining, and/or quantifying the expression of STn may be used in diagnosing conditions associated with the expression of STn, such as cancer. Thus, the humanized mAbs, the fragments of the present invention or the conjugates of the present invention are for use in diagnosing, monitoring the progression of cancer, or monitoring and estimating the effectiveness of treatment of cancer. The term “monitoring cancer” encompasses the term monitoring the progression of cancer and monitoring the effectiveness of treatment of cancer. In some embodiments, the present invention provides a method of diagnosing, assessing the severity or staging of a proliferative disease such as cancer in a subject, the method comprises detecting the presence or expression of STn in a biological sample of the subject using at least one antibody, antibody fragment or conjugate of the present invention or the composition comprising same. According to some embodiments, the antibodies or fragments thereof are conjugated or labeled. According to some embodiments, the method comprises quantitatively comparing the level of expression of the STn glycan in a subject to a reference expression level of e.g. healthy subjects. According to another embodiment, the method comprises comparing the measured amount of STn in the biological sample of the subject to a threshold. According to some embodiments, a change in expression of STn in comparison to healthy subjects indicates the presence of cancer. According to some embodiments, overexpression of the STn correlates with cancer. Thus, in some embodiments, detecting STn expression level above the reference value obtained from healthy subjects correlates with the presence of cancer. According to some embodiments, the cancer is as described hereinabove. Thus, in some embodiments, detecting STn expression level above the reference value or a threshold correlates with the presence of cancer. According to one embodiment, the present invention provides a method for diagnosing cancer in a subject, the method comprises contacting a biological sample of the subject with the monoclonal antibodies or antibody fragments or conjugates of the present invention, preferably under conditions which allow immunocomplexes formation, and assessing the amount of STn in the sample, wherein the cancer overexpresses STn glycan, wherein method comprises comparing the assessed amount of STn in the sample to a threshold or to a reference, wherein the reference is the level of STn in the sample of healthy subjects, and wherein the amount of the STn in the sample above the reference of the threshold is indicative of the malignancy overexpressing STn. According to some embodiments, the cancer is selected from endometrial carcinoma, ovarian carcinoma, prostate c adenocarcinoma, seminoma, diffuse type gastric adenocarcinoma, pancreatic and colon adenocarcinomas, lung adenocarcinoma and mantle cell lymphoma. The term “biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. Additionally, the term may encompass circulating tumor or other cells. The term specifically encompasses a clinical sample and further includes cells in cell culture, cell supernatants, cell lysates, serum, plasma, urine, amniotic fluid, biological fluids including aqueous humor and vitreous for eyes samples, and tissue samples. The term also encompasses samples that have been manipulated in any way after procurement, such as treatment with reagents, solubilization, or enrichment for certain components.

According to any one of the above embodiments, the method comprises detecting STn in the sample, e.g. biological sample. The method comprises contacting the biological sample with the antibody or the fragment of the present invention. According to some embodiments, the antibody or the fragment are marked, tagged or labeled. According to other embodiments, secondary antibodies may be used to determine the level of binging of the antibody of the present invention or the fragment to the biological sample of its components. According to some embodiments, any known methods for determining and quantifying binding of an antibody or a fragment thereof to its target may be used. According to some embodiments, detecting comprises quantifying the amount of the STn. According to some embodiment, the method comprises a comparison of the content of the STn in a biological sample obtained from a subject to the control, i.e. comparing to the content of STn in the comparable biological sample of healthy subjects. According to some embodiments, the monitoring method comprises comparing STn content in a sample obtained from a subject at different times and assessing the propagation (i.e. monitoring) of the disease and/or effectiveness of treatment. According to some embodiments, the present invention provides a method of detection of STn in a tissue culture, in a tissue or in a section obtained from a subject.

According to any one of the above embodiments, the method further comprises consulting or providing recommendations regarding the treatment of the disease or condition or providing the treatment of the disease, such as cancer. According to any one of the above embodiments, the method further comprises treating cancer. Thus, according to some embodiment, the present invention provides a method of treating a malignancy overexpressing STn in a subject in need thereof, the method comprising: (a) diagnosing the malignancy in the subject by the methods of the present invention, and (b) treating the malignancy when the malignancy is detected.

According to some embodiments, the present invention provides a method of monitoring the treatment of a STn-overexpressing cancer, the method comprises determining a level of STn in a subject in need thereof as described above before and after treating the cancer, wherein a decrease in said level following said treating is indicative of efficacious treatment.

The methods of determining or quantifying the expression of the STn according to any one of the above embodiments comprise contacting a biological sample with an antibody or antibody fragment or conjugate and measuring the level of complex formation. Determining and quantifying methods may be performed in-vitro or ex-vivo. The antibodies according to the present invention may be also used to configure screening methods. For example, an enzyme-linked immunosorbent assay (ELISA), or a radioimmunoassay (RIA), as well as methods such as IHC or FACS, can be constructed for measuring levels of secreted or cell-associated STn glycan using the antibodies of the present invention and methods known in the art. According to some embodiments, the method for detecting or quantifying the presence of STn expressed on cells comprises the steps of:
incubating a biological sample with the humanized antibodies or antibody fragments of the present invention comprising at least an antigen-binding portion; and
detecting the bound STn using a detectable probe.

According to some embodiments, the method further comprises the steps of:
comparing the amount of (ii) to a standard curve obtained from a reference sample containing a known amount of STn; and
calculating the amount of the STn in the sample from the standard curve.

According to some particular embodiments, the sample is a body fluid.

According to some embodiments, the method is performed in-vitro or ex-vivo.

According to another aspect, the present invention provides a kit for detecting cancer, wherein the kit comprises antibodies, antibody fragments or conjugates of the present invention and means for detecting the amount of the antibodies, antibody fragments or conjugates bound to cells of the biological sample. According to some embodiments, the kit is a diagnostic kit. According to some embodiments, the kit further comprises means for detecting and quantifying the formation of the complex of the antibodies, antibody fragments or conjugates with STn glycan. According to some embodiments, the kit further comprises reference levels of the STn glycan in healthy subjects. According to some embodiments, the kit comprises means for performing analysis in a plurality of times and means for comparison of the results obtained in the measurement.

By using the kit, a person skilled in the art may measure the amount of STn glycan present in the biological sample and compare it to a reference. According to some embodiments, the kit is for monitoring the treatment or development of the cancer, and the kit comprises means for measurement of the amount of STn glycan in biological samples two or more times. In case of monitoring the treatment of development of the cancer the reference may be the previously taken biological sample of the subject or the results obtained in the previous measurement.

According to some embodiments, the isolated monoclonal antibody, the humanized isolated monoclonal antibody or a fragment thereof or the conjugate thereof is immobilized to a solid support. According to some embodiments, the humanized isolated monoclonal antibody or a fragment thereof or the conjugate thereof is attached to a detectable moiety.

According to some embodiments, the Abs, fragments or conjugates are immobilized on a solid surface. Any solid surface may be used such as chip or microarray. According to some embodiments, the solid phase is a membrane. According to another embodiment, the solid phase is a polymer. Non limiting examples of solid phases are nitrocellulose, polyvinylidene fluoride (PVDF); hydrophobic (Charge-modified) nylon and polyethersulfone (PESU). Alternatively, the solid phase may be a woven meshes, synthetic nonwovens, cellulose and glass fiber.

According to alternative embodiments, the Abs, fragments or conjugates are dissolved in a solvent. According to some embodiments, the Abs, fragments or conjugates are linked to beads.

According to some embodiments, the kit comprises means for quantifying the amount of antibodies bound STn glycan.

According to another aspect, the present invention provides a method of preparation of the T-cells of the present invention. All terms, embodiments and definitions defined in any one of the above aspects apply and are encompassed herein as well. According to one embodiment, the present invention provides a method of preparation of T-cells genetically modified to express the CARs of the present invention. According to some embodiments, the said method comprises transfecting of T-cells with the DNA construct of the present invention.

The terms “transfection”, “transduction”, “transfecting” or “transducing” can be used interchangeably and are defined as a process of introducing a nucleic acid molecule to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection, any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

According to one embodiment, the T-cells are CD4+ T-cells. According to another embodiment, the T-cells are CD8+ cells. According to one embodiment, the T-cells are a combination of CD4+ and CD8+ cells.

According to one embodiment, the method comprises transducing T cells with at least one DNA construct encoding the CAR comprising an amino acid sequence selected from SEQ ID NO: 41 and 42. According to some embodiments, the DNA construct comprises a nucleic acid sequence selected from SEQ ID NO: 43, 44 and a variant thereof as defined in the present invention.

According to any one of the above embodiments, the transduction is performed using a viral vector selected from retroviral, adenoviral, lentiviral and adeno-associated viral vectors.

According to some embodiments, the vector may contain an optional marker suitable for use in the identification of the transformed cells.

The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

### Methods

Humanization and Cloning of mRAQ

The DNA and amino acid sequences of the variable heavy (V_{H}) and the variable light (V_{L}) regions of the mouse-derived anti Sialyl Tn antibody (denoted as mRA0) were compared to human germline sequences in the human immunoglobulin database by the IgBlast tool.

Mouse RA0 (mRA0) VH and VL sequences are set for the in SEQ ID NO: 1 and 2, respectively.

The blast of VH DNA results in the best fit to the IGHV2-70*18 germline (>67% identity). Blast of VH amino acid results in the best fit to the IGHV4-4*08 germline (>65% identity). This germline sequences database does not cover the framework 4 (FR4) region, therefore IGHJ sequence was screened in IMGT database for common human VH FR4 with sequence similarity to the VH FR4 of mRA0 antibodies. Out of the three different alleles of the highest sequence similarity, IGHJ1*01 sequence was selected as the basis for VH FR4 humanization. For VL humanization the DNA sequence of mRA0 VL was used, which resulted in the best fit to the IGKV3-11*01 germline (>70% identity). Screening of IGKJ sequences in IMGT database for common human VL FR4 with sequence similarity to the VL FR4 of mRA0 antibodies revealed that IGKJ2*01 sequence has the highest similarity, thus it is selected as the basis for VL FR4 humanization. For full antibody humanization, the framework sequences were then mutated based on the selected germline sequences, while the CDRs were preserved based on the corresponding mRA0 antibodies to yield the humanized RA0 (HuRA0) antibodies. These antibodies were termed HuRA0-V1 and HuRA0-V2. HuRA0-V1 comprise a VH domain which frameworks were derived from IGHV2-70*18 germline to obtain VH domain having amino acid sequence SEQ ID NO: 25 and a VL domain having an amino acid sequence SEQ ID NO: 26. HuRA0-V2 comprises a VH domain, in which VH frameworks were derived from IGHV4-4*08 germline to obtain VH domain having amino acid sequence SEQ ID NO: 27, and VL domain as in HuRA0-V2. Additional versions of HuRA0 were generated. Some residues of closely related human germline sequences were positioned in the antibody β-strands regions. Since these ß-strands likely serve as the scaffold for CDRs it was hypothesized that it might interfere the antibody binding pocket. Therefore, selected residues in VH (3, 5, 19, 20, 24, 68, 70, 79, 81, 82, 83, 84, 86; according to Kabat) were maintained as in the original mRA0. In addition, residues 30, 49, 93, 94 are flanking regions around the CDRs so they were also conserved as in the original mRA0 to preserve proper CDR presentation by these residues. The obtained VH domain has an amino acid sequence SEQ ID NO: 28. For the VL domain, two sequences were generated; in the first version, residues 19, 21, 22, 45, 46, 47, 70, 71, 72, 80, 83, 85 (according to Kabat) were maintained as in the original mRA0 to obtain an amino acid sequence SEQ ID NO: 29. In the second version, residues 45, 46, 47, 80, 83, 85 (according to Kabat) were maintained as in the original mRA0 to obtain an amino acid sequence SEQ ID NO: 30. The combination of VH (SEQ ID NO: 28) with the first VL version sequence (SEQ ID NO: 29) is termed HuRA0-V7, while the combination of the VH domain having amino acid sequence SEQ ID NO: 28 and the second VL version (SEQ ID NO: 30) is termed HuRA0-V8. mRA0 and HuRA0 scFvs were formed by binding VH and VL domain with (G₄S)₃ linker (DNA seq: ggaggtggcggtagcggaggcggcggttctggaggtggcgggagc; Amino acids seq: GGGGSGGGGSGGGGS) is synthesized by Integrated DNA Technologies Inc. As such two scFv were produced HuRA0 V7 scFv having an amino acid sequence SEQ ID NO: 31 and HuRA0 V8 scFv having an amino acid sequence SEQ ID NO: 32.

Expression of mRA0 and HuRA0 Single-Chain Fv (scFv) Fragments on Yeast Cells

To obtain yeast cells with surface expression of the mouse and humanized antibody clones, the single-chain Fv (scFv) fragments of mRA0 and HuRA0 (comprising the VH and VL domains combined by a linker) were cloned into the YSD pETCON2-based system.

Corresponding scFv fragments with flanking regions homologous to pETCON2 VH/VL plasmids were synthesized (Integrated DNA Technologies Inc.; IDT, Israel). scFv fragments were amplified by PCR, reaction was made in Q5 reaction buffer, with 10 ng of scFv template, 200 μM each dNTP, 1 U Q5 hot start high fidelity DNA polymerase (New England Biolabs), 500 nM each primer complete volume to 50 μl with PCR grade water. PCR conditions are 95° C. for 2 min followed by 12 cycles of 95° C. for 30 s, 55° C. for 30 s, 72° C. for 60 s, and final incubation of 72° C. for 5 min. Each amplified fragment was purified by Wizard SV Gel and PCR clean-up system. EBY100 yeast cells were transformed with scFv by LiAc/SS Carrier DNA/peg method, as described in Gictz and Schiestl (2007, Nat Protoc. 2007; 2(1):31-4), using 150-250 ng of Ndel and BamHI (Thermo Scientific) digested pETCON2 plasmid and 150-250 ng of gel-purified scFv in a 1:1 ratio. Following recovery, the yeast cells were plated on a synthetic defined media (SD) lacking Tryptophan (Trp) [SD-Trp plates; 2% glucose (Sigma), 0.67% yeast nitrogen base w/o amino acids (BD), 0.54% Na2HPO4 (Sigma), 0.86% NaH2PO4 (Sigma) and 0.192% yeast synthetic drop-out medium supplements without Trp (Sigma)], then incubated at 30° C. Two days later, single colonies were picked and cultured in SD-Trp liquid media, and plasmids were purified from yeast cells using Zymoprep Yeast Plasmid Miniprep II (Zymo Research) according to manufacturer's instructions. To validate the sequences, plasmids are electroporated into XL1 Escherichia coli, plasmids are purified by NucleoSpin Plasmid EasyPure (Macherey-Nagel) and sequences analyzed at Tel Aviv University core facility.

### Specificity Analysis of Yeast Cells Expressing Surface-scFv

scFv-mRA0 and scFv-HuRA0 yeast variants were cultured in SD-Trp at 30° C., passaged 1:10 each day for three days, then scFv are triggered to be expressed by transfer to a synthetic galactose (SG) based media [SG-Trp media: 2% galactose (Sigma), 0.2% glucose, 0.67% yeast nitrogen base w/o amino acids, 0.54% Na₂HPO₄, 0.86% NaH₂PO₄, and 0.192% yeast synthetic drop-out medium supplements without Trp], then grown overnight at 20° C. Then 5×10⁶ yeast cells were washed with 1 ml assay buffer (PBS, 0.5% ovalbumin), incubated with 1 M STn-PAA-Biotin or 1 μM Tn-PAA-Biotin antigens together with 1:50 diluted mouse-anti-c-Myc (4 μg/ml) diluted in assay buffer for 1 h at RT with rotation. Cells were washed with 1 ml ice cold assay buffer, then incubated for 40 min on ice with APC-streptavidin and Alexa-Fluor-488-goat-anti-mouse IgG1 diluted 1:50 (10 μg/ml) and 1:200 (10 μg/ml) respectively in assay buffer. Cells were washed with 1 ml ice cold PBS, then resuspended in 500 μl PBS. Cell fluorescence was measured by CytoFLEX flow cytometry (Beckman Coulter) and analyzed with Kaluza analysis software.

Immunogenicity Analysis of scFv-mRA0 and scFv-HuRA0

Immunogenicity of humanized antibody clones was evaluated by analysis of scFv recognition by pooled human IgG obtained from thousands of human donors (IVIg; Gamma Gard). For this purpose, IVIg was first pre-cleared from anti-yeast reactivity by serial incubations with yeast cells, then the binding to scFv-expressing yeast cells was examined. Uninduced HuNative RA0 yeasts cells grown in SD-Trp at 30° C. were divided into 9 different Eppendorf tubes with 5×10⁶ cells in each. Cells are washed twice with 1 ml PBS, then supernatant is removed. For anti-yeast adsorption, yeast cells in the first tube are resuspended in 1 ml of 68 mg/ml IVIg, followed by 10 min with rotation of 30 rpm at RT. Yeast-IVIg mixture is centrifuged at 10,000×g for 1 min, and supernatant with unbound antibodies was transferred into a fresh yeast pellet tube for the second cycle of antibody adsorption as described, and this was repeated for a total of nine incubations, thus decreasing the amount of anti-yeast antibodies in the IVIg resulting in a “yeast-purified IVIg” pooled human IgG. Subsequently, scFv-mRA0 and scFv-HuRA0 yeast variants were induced to express scFv as indicated (by transfer to SG-Trp media at 20° C.), then 5×10⁶ yeast cells were washed with 1 ml assay buffer (PBS, 0.5% ovalbumin), and incubated with 50 ng/μl yeast-purified IVIg in assay buffer for 45 min at RT with rotation. Cells were washed with 1 ml ice-cold assay buffer, and then incubated for 45 min on ice with 1:50 diluted mouse-anti-c-Myc (4 μg/ml) in assay buffer. Cells were washed with 1 ml ice cold assay buffer, then incubated for 40 min with Cy3-anti-human Fc specific and Alexa-Fluor-488-goat-anti-mouse IgG1 diluted 1:100 (15 μg/ml) and 1:200 (10 μg/ml) respectively in assay buffer. Cells are washed with 1 ml ice-cold PBS, then resuspended in 500 μl PBS for flow cytometry analysis.

Gibson Assembly of Full-Length mRA0 and HuRA0 Antibodies Expression Plasmids

Variable heavy and variable light fragments of mRA0 and HuRA0 were amplified by PCR. Reaction is made in Q5 reaction buffer, with 1 μl of plasmid DNA template purified from E. coli, 200 μM each dNTP, 1 U Q5 hot start high fidelity DNA polymerase (New England Biolabs), 500 nM each primer complete volume to 50 μl with PCR grade water. PCR conditions are 95° C. for 2 min followed by 30 cycles of 95° C. for 30 s, 61° C. for 60 s, 72° C. for 60 s, and final incubation of 72° C. for 5 min. To remove template segments, the PCR product was supplemented with 6 μl of 10× FastDigest Green Buffer, 1 μl FastDigest DpnI (Thermo Scientific), and completed the volume to 60 μl with PCR grade water, then incubated at 37° C. for 1 h. PCR digested fragments are purified from agarose gel by Zymoclean Gel DNA Recovery Kit (Zymo Research). Heavy and light chain full IgG p3BNC expression plasmids were divided into three parts for PCR amplification, variable region, left arm and right arm. Left and right arms of heavy and light p3BNC plasmids are amplified, digested and purified. Of each fragment (variable region, right arm and left arm), 25 ng are taken for Gibson assembly. Reaction is made in isothermal reaction buffer containing 3.75% PEG 8000, 75 mM Tris-HCl pH 7.5, 7.5 mM MgCl2, 7.5 mM DTT, 0.15 mM of each dNTP and 0.75 mM NAD. To this buffer 0.04 U T5 exonuclease (NEB), 0.25 U Phusion polymerase (NEB) and 40 U Taq DNA ligase (NEB) were added and ligation is made at 50° ° C. for 1 h. Plasmids were electroporated into XL1 Escherichia coli, to validate the sequence and for production p3BNC expression plasmids.

Expression and Purification of Full-Length mRA0 and HuRA0 IgG Antibodies

Human embryonic kidney 293A cells were used to produce whole antibodies clones from the p3BNC expression plasmids template transfected with polyethylenimine reagent (PEI; Polysciences), as described (Amon et al., 2018). Antibodies were purified using protein A (GE healthcare) and concentrations were determined by BCA assay (Pierce).

### Sialoglycan Microarray Fabrication.

Arrays were fabricated with NanoPrint LM-60 Microarray Printer (Arrayit) on epoxide-derivatized slides (Corning 40044) with 16 sub-array blocks on each slide. Glycoconjugates were distributed into one 384-well source plate using 4 replicate wells per sample and 8 μl per well (Version 2.0). Each glycoconjugate (see Table 1) was prepared at 100 μM in an optimized print buffer (300 mM phosphate buffer, pH 8.4). To monitor printing quality, replicate-wells of human IgG (80, 40, 20, 10, 5, 0.25 ng/μl in PBS+10% glycerol) and AlexaFlour-555-Hydraside (Invitrogen A20501MP, at 1 ng/μl in 178 mM phosphate buffer, pH 5.5) were used for each printing run. The arrays were printed with four 946MP3 pins (5 μm tip, 0.25 μl sample channel, ˜100 μm spot diameter; Arrayit). Each block (sub-array) had 20 spots/row, 20 columns with spot to spot spacing of 275 μm. The humidity level in the arraying chamber was maintained at about 70% during printing. Printed slides are left on arrayer deck over-night, allowing humidity to drop to ambient levels (40-45%). Next, slides were packed, vacuum-sealed and stored at room temperature (RT) until used.

TABLE 1
List of glycans fabricated on glycan microarrays.

| ID | Structure |
|---|---|
| 1 | Neu5,9Ac2α3Galβ4GlcNAcβO(CH₂)₂CH₂NH₂ |
| 2 | Neu5Gc9Acα3Galβ4GlcNAcβO(CH₂)₂CH₂NH₂ |
| 3 | Neu5,9Ac2α6Galβ4GlcNAcβO(CH₂)₂CH₂NH₂ |
| 4 | Neu5Gc9Acα6Galβ4GlcNAcβO(CH₂)₂CH₂NH₂ |
| 5 | Neu5Acα6GalNAcαO(CH₂)₂CH₂NH₂ |
| 6 | Neu5Gcα6GalNAcαO(CH₂)₂CH₂NH₂ |
| 7 | Neu5,9Ac2α3Galβ3GlcNAcβO(CH₂)₂CH₂NH₂ |
| 8 | Neu5Gc9Acα3Galβ3GlcNAcβO(CH₂)₂CH₂NH₂ |
| 9 | Neu5,9Ac2α3Galβ3GalNAcαO(CH₂)₂CH₂NH₂ |
| 10 | Neu5Gc9Acα3Galβ3GalNAcαO(CH₂)₂CH₂NH₂ |
| 11 | Neu5Acα3Galβ4GlcNAcβO(CH₂)₂CH₂NH₂ |
| 12 | Neu5Gcα3Galβ4GlcNAcβO(CH₂)₂CH₂NH₂ |
| 13 | Neu5Acα3Galβ3GlcNAcβO(CH₂)₂CH₂NH₂ |
| 14 | Neu5Gcα3Galβ3GlcNAcβO(CH₂)₂CH₂NH₂ |
| 15 | Neu5Acα3Galβ3GalNAcαO(CH₂)₂CH₂NH₂ |
| 16 | Neu5Gcα3Galβ3GalNAcαO(CH₂)₂CH₂NH₂ |
| 17 | Neu5Acα6Galβ4GlcNAcβO(CH₂)₂CH₂NH₂ |
| 18 | Neu5Gcα6Galβ4GlcNAcβO(CH₂)₂CH₂NH₂ |
| 19 | Neu5Acα6Galβ4GlcβO(CH₂)₂CH₂NH₂ |
| 20 | Neu5Gcα6Galβ4GlcβO(CH₂)₂CH₂NH₂ |
| 21 | Neu5Acα3Galβ4GlcβO(CH₂)₂CH₂NH₂ |
| 22 | Neu5Gcα3Galβ4GlcβO(CH₂)₂CH₂NH₂ |
| 23 | Neu5,9Ac2α6GalNAcαO(CH₂)₂CH₂NH₂ |
| 24 | Neu5Gc9Acα6GalNAcαO(CH₂)₂CH₂NH₂ |
| 25 | Neu5Acα3GalβO(CH₂)₂CH₂NH₂ |
| 26 | Neu5Gcα3GalβO(CH₂)₂CH₂NH₂ |
| 27 | Neu5Acα6GalβO(CH₂)₂CH₂NH₂ |
| 28 | Neu5Gcα6GalβO(CH₂)₂CH₂NH₂ |
| 29 | Neu5,9Ac2α3GalβO(CH₂)₂CH₂NH₂ |
| 30 | Neu5Gc9Acα3GalβO(CH₂)₂CH₂NH₂ |
| 31 | Neu5,9Ac2α6GalβO(CH₂)₂CH₂NH₂ |
| 32 | Neu5Gc9Acα6GalβO(CH₂)₂CH₂NH₂ |
| 33 | Neu5Acα3Galβ3GalNAcβO(CH₂)₂CH₂NH₂ |
| 34 | Neu5Gcα3Galβ3GalNAcβO(CH₂)₂CH₂NH₂ |
| 35 | Neu5,9Ac₂α3Galβ3GalNAcβO(CH₂)₂CH₂NH₂ |
| 36 | Neu5Gc9Acα3Galβ3GalNAcβO(CH₂)₂CH₂NH₂ |
| 37 | Neu5,9Ac₂α6Galβ4GlcβO(CH₂)₂CH₂NH₂ |
| 38 | Neu5Gc9Ac6Galβ4GlcβO(CH₂)₂CH₂NH₂ |
| 39 | Neu5,9Ac2α3Galβ4GlcβO(CH₂)₂CH₂NH₂ |
| 40 | Neu5Gc9Ac3Galβ4GlcβO(CH₂)₂CH₂NH₂ |
| 41 | Neu5Acα8Neu5Acα3Galβ4GlcβO(CH₂)₂CH₂NH₂ |
| 42 | Neu5Acα8Neu5Acα8Neu5Acα3Galβ4GlcβO(CH₂)₂CH₂NH₂ |
| 55 | Neu5Acα3Galβ4(Fucα3)GlcNAcβO(CH₂)₂CH₂NH₂ |
| 56 | Neu5Gcα3Galβ4(Fucα3)GlcNAcβO(CH₂)₂CH₂NH₂ |
| 57 | Neu5Acα3Galβ4(Fucα3)GlcNAc6SβO(CH₂)₂CH₂NH₂ |
| 58 | Neu5Gcα3Galβ4(Fucα3)GlcNAc6SβO(CH₂)₂CH₂NH₂ |
| 59 | Galβ3GlcNAcβ3Galβ4GlcβO(CH₂)₂CH₂NH₂ |
| 60 | Neu5Acα3Galβ3GlcNAcβ3Galβ4GlcβO(CH₂)₂CH₂NH₂ |
| 61 | Neu5Gcα3Galβ3GlcNAcβ3Galβ4GlcβO(CH₂)₂CH₂NH₂ |
| 62 | Neu5Acα3Galβ4GlcNAc6SβO(CH₂)₂CH₂NH₂ |
| 63 | Neu5Gcα3Galβ4GlcNAc6SβO(CH₂)₂CH₂NH₂ |
| 64 | Neu5Acα8Neu5Acα3Galβ4GlcβO(CH₂)3NHCOCH₂(OCH₂CH₂)6NH₂ |
| 65 | Neu5Acα8Neu5Acα8Neu5Acα3Galβ4GlcβO(CH₂)3NHCOCH₂(OCH₂CH₂)6NH₂ |
| 66 | Neu5Acα6(Neu5Acα3)Galβ4GlcβO(CH₂)₂CH₂NH₂ |
| 67 | Neu5Acα6(Neu5Gcα3)Galβ4GlcβO(CH₂)₂CH₂NH₂ |
| 68 | Neu5Acα6(Kdnα3)Galβ4GlcβO(CH₂)₂CH₂NH₂ |
| 69 | Neu5Gcα8Neu5Acα3Galβ4GlcβO(CH₂)₂CH₂NH₂ |
| 70 | Kdnα8Neu5Acα3Galβ4GlcβO(CH₂)₂CH₂NH₂ |
| 71 | Neu5Acα8Kdnα6Galβ4GlcβO(CH₂)₂CH₂NH₂ |
| 72 | Neu5Acα8Neu5Gcα3Galβ4GlcβO(CH₂)₂CH₂NH₂ |
| 73 | Neu5Acα8Neu5Gcα6Galβ4GlcβO(CH₂)₂CH₂NH₂ |
| 74 | Kdnα8Neu5Gcα3Galβ4GlcβO(CH₂)₂CH₂NH₂ |
| 75 | Neu5Gcα8Neu5Gcα3Galβ4GlcβO(CH₂)₂CH₂NH₂ |
| 76 | Neu5Acα8Neu5Acα6Galβ4GlcβO(CH₂)₂CH₂NH₂ |
| 77 | Neu5GcMeα8Neu5Acα3Galβ4GlcβO(CH₂)₂CH₂NH₂ |
| 78 | Galα3Galβ4GlcNAcβO(CH₂)₂CH₂NH₂ |
| 79 | Galβ3GalNAcαO(CH₂)₂CH₂NH₂ |
| 80 | Galβ4(Fucα3)GlcNAcβO(CH₂)₂CH₂NH₂ (LexβProNH₂) |
| 81 | Neu5Acα8Neu5Acα3Galβ4GlcO(CH₂)₂CH₂NH₂ (GD3) |
| 82 | Neu5Acα8Neu5Acα3(GalNAcβ4)Galβ4GlcO(CH₂)₂CH₂NH₂ (GD₂) |
| 83 | Neu5Acα3Galβ3(Fucα4)GlcNAcβO(CH₂)₂CH₂NH₂ |
| 84 | Galβ3(Fucα4)GlcNAcβO(CH₂)₂CH₂NH₂ |
| 85 | Fucα₂Galβ3(Fucα4)GlcNAcβO(CH₂)₂CH₂NH₂ |
| 86 | Neu5Gcα3Galβ3(Fucα4)GlcNAcβO(CH₂)₂CH₂NH₂ |
| 87 | Neu5,9Ac2α3Galβ3(Fucα4)GlcNAcβO(CH₂)₂CH₂NH₂ |
| 88 | Neu9Ac5Gcα3Galβ3(Fucα4)GlcNAcβO(CH₂)₂CH₂NH₂ |
| 89 | Neu5Acα8Neu5Acα3(Neu5Acα3Galβ3GalNAcβ4)Galβ4GlcβO(CH₂)₂CH₂NH₂ |
| 90 | Galβ4GlcNAcβ3Galβ4GlcβO(CH₂)₂CH₂NH₂ |
| 91 | Neu5Acα3Galβ4GlcNAcβ3Galβ4GlcβO(CH₂)₂CH₂NH₂ |
| 92 | Neu5Gcα3Galβ4GlcNAcβ3Galβ4GlcβO(CH₂)₂CH₂NH₂ |
| 93 | Neu5,9Ac2α3Galβ4GlcNAcβ3Galβ4GlcβO(CH₂)₂CH₂NH₂ |
| 94 | Neu9Ac5Gcα3Galβ4GlcNAcβ3Galβ4GlcβO(CH₂)₂CH₂NH₂ |
| 95 | Neu5Acα6Galβ4GlcNAcβ3Galβ4GlcβO(CH₂)₂CH₂NH₂ |

### Sialoglycan Microarray Binding Assay.

Slides were developed and analyzed as previously described in Amon et al., (Cancers (Basel). 2020 Sep. 30; 12(10):2824. doi: 10.3390/cancers12102824. PMID: 33007970) with some modifications. Slides were rehydrated with dH₂O and incubated for 30 min in a staining dish with 50° C. pre-warmed ethanolamine (0.05 M) in Tris-HCl (0.1 M, pH 9.0) to block the remaining reactive epoxy groups on the slide surface, then washed with 50° C. pre-warmed dH₂O. Slides were centrifuged at 200×g for three min then fitted with ProPlate™ Multi-Array 16-well slide module (Invitrogen) to divide into the sub-arrays (blocks). Slides were washed with PBST (0.1% Tween 20), aspirated and blocked with 200 μl/sub-array of blocking buffer (PBS/OVA, 1% w/v ovalbumin, in PBS, pH 7.3) for 1 hour at RT with gentle shaking. Next, the blocking solution was aspirated and 100 μl/block of purified HuRA0 antibody in 0.16 ng/μl diluted in PBS/OVA are incubated with gentle shaking for 2 hours at RT. Slides were washed three times with PBST, then with PBS for 2 min. Bound antibodies were detected by incubating with secondary detection diluted in PBS, 200 μl/block at RT for 1 hour, Cy3-goat anti-mouse IgG 1.5 μg/ml (Jackson Immunoresearch). Slides were washed three times with PBST then with PBS for 10 min followed by removal from ProPlate™ Multi-Array slide module and immediately dipping in a staining dish with dH₂O for 10 min with shaking, then centrifuged at 200×g for 3 min. Dry slides were immediately scanned.

### Array Slide Processing.

Processed slides were scanned and analyzed as described at 10 μm resolution with a Genepix 4000B microarray scanner (Molecular Devices) using 300 gain. Image analysis was carried out with Genepix Pro 6.0 analysis software (Molecular Devices). Spots were defined as circular features with a variable radius as determined by the Genepix scanning software. Local background subtraction was performed.

### Enzyme-Linked Immunosorbent Assay (ELISA)

Binding of HuRA0 anti-STn hIgG to STn was tested by ELISA. Costar 96-well were coated overnight at 4° C. with 0.25 μg STn-PAA-Biotin (Glycotech) in coating buffer (50 mM sodium carbonate-bicarbonate buffer, pH 9.5). HuRA0 antibodies at 0.2 ng/μl are pre-incubated with free glycans, STn-PAA-Biotin, Neu5Acα2-3GalNAcα-PAA-Biotin, Tn-PAA-Biotin, SLc^{a}-PAA-Biotin (glycotech), for 2 h on ice. Wells are blocked for 1 h at RT with blocking buffer (PBS pH 7.3, 1% chicken ovalbumin (Sigma); PBS/OVA). After removal of blocking buffer, HuRA0-glycan mixture were added to triplicate wells at 100 μl/well then incubated at RT for 2 h. Wells were washed three times with PBST (PBS pH 7.3, 0.1% Tween-20), and the detection antibody was then added (100 μl/well, 1:7000 HRP-goat-anti-human IgG (H+L) diluted in PBS and incubated for 1 h at RT. After washing three times with PBST, wells were developed with 0.5 mg/ml O-phenylenediamine in citrate-PO₄ buffer, pH 5.5. The reaction was stopped with H₂SO₄ and absorbance was measured at a 490 nm wavelength on a SpectraMax M3 (Molecular Devices).

### Tissue Microarray Immunohistochemical Staining

The cloned HuRA0 human IgG1 antibodies were biotinylated using the EZ-Link biotinylation Kit (Micro Sulfo-NHS-SS-Biotin; Pierce, Rockford, IL) according to the manufacturer's instructions. Then, human cancers tissue microarray (TMA) slides (BioSB CA, USA) consisting of twenty-three 2 mm cores formalin-fixed paraffin-embedded tissues was stained with this Bio-HuRA0-hIgG antibody. For this purpose, the slides were first deparaffinated by incubation in xylene (Merck) for 15 min twice, then rehydrated by sequential 2 min washes with a decreased percentage of ethanol in double distilled H₂O solution (100%, 95%, 90%, 80%, 70%, 50%, DDW), then washed twice in DDW. For antigen unmasking, slides were incubated for 15 min with 95° C. pre-heated HIER T-EDTA buffer pH 9 (Zymo), then transferred to DDW for additional 15 min, followed by rinsing in PBS pH 7.4 once. Slides were then blocked for one hour at room temperature (RT) by incubating with blocking solution (PBS pH 7.4, 0.1% Tween, 1% chicken ovalbumin [Sigma]). Biotin/avidin blocking was performed using a kit (Zotal), according to the manufacturer's instructions. Slides were rinsed briefly with PBS, then fixed with 4% paraformaldehyde (PFA) for 10 min in RT, washed with PBST (PBS pH 7.4, 0.1% Tween) for 1 min, and incubated with 10 ng/μl Bio-HuRA0-hIgG overnight at 4° C. in a humidified chamber. The next day, slides were washed in PBST for 5 min, twice, then incubated with freshly prepared 0.3% H₂O₂ in PBS for 15 min. After one wash with PBS pH7.4, slides were incubated with 1 μg/ml HRP-streptavidin in PBS (Jackson) for 30 minutes at RT, followed by three washes with PBS 5 min each, then developed with the substrate (3,3′-diaminobenzidine tetrahydrochloride; DAB) for 3 min, followed by washing once with DDW for 1 min and mounting with PermaMounter (Bio-SB). Slides are screened with Nikon eclipse Ti microscope at ×10 magnification.

### Cell Lines and Culture Cell Lines

293T human embryonic cells (ATCC; CRL-1573), FaDu pharynx squamous cell carcinoma cells (ATCC; HTB43), Raji, MEG-01, Capan-2, and packaging cell lines and PG13 (ATCC; CRL-10686) were cultured in DMEM supplemented with 10% fetal calf serum (FCS), 2 mM glutamine and 1 mM sodium pyruvate. Mouse lymphocytes were cultured in RPMI-1640 (Biological Industries) supplemented with 10% FCS, 2 mM glutamine. All media was supplemented with a mixed antibiotic solution containing penicillin (100 U/ml), streptomycin (100 μg/ml) and neomycin (10 μg/ml) (Bio-Lab). B16F10 was a kind gift from Professor Dan Peer at Tel Aviv University, cultured in DMEM supplemented with 10% fetal calf serum (FCS), 2 mM glutamine and a mixed antibiotic solution containing penicillin (100 U/ml), streptomycin (100 μg/ml). The cells were incubated in a humidified 37° ° C. incubator with 5% CO₂, except for the PG13, which are kept with 7.5% CO₂. All cells are verified to lack mycoplasma by PCR (HyLabs). The cells were frozen at low passage, and the number of passages after thawing was recorded. Cells were maintained in the culture for no longer than 4 weeks, which corresponds to approximately 12 passages.

### Construction of Chimeric Antigen Receptor (CAR) and Retroviral Vector Production

The CAR constructs contained a leader signal peptide, HuRA0 scFv (VH connected to the VL through 3×G₄S spacer), strep-tag, 2×G₄S spacer, human CD28 (hCD28 cytoplasmic, transmembrane and co-stimulation domains) followed by the human FcγR ITAM signaling domain. Additional CAR constructs were prepared without strep-tag. Such CAR constructs comprise (i) a leader signal peptide, HuRA0 scFv 2×G₄S spacer, human CD28 (hCD28 cytoplasmic, transmembrane and co-stimulation domains) followed by the human FcγR ITAM signaling domain or (ii) a leader signal peptide, HuRA0 scFv (VH connected to the VL through 3×G₄S spacer), human CD28 (hCD28 cytoplasmic, transmembrane and co-stimulation domains) followed by the human FcγR or CD3zeta ITAM signaling domain.

The sequence was cloned into the retroviral vector pMSGV1.

In order to produce a stable packaging cell line, 293T cells were co-transfected with retroviral vector plasmids (Gag-Pol) and the plasmid of interest (pMSGV1-CAR T) using CaPO₄, as described (Elinav et al., 2009). Supernatants containing the retrovirus were collected 16 hours later and used to stably transduce the amphotropic PG13 packaging cells. Cells were sorted by FACSort flow Cytometer (BD PharMingen) to achieve 100% HuRA0-CAR⁺-PG13 expressing cells, re-grown and frozen at −80° C.

### T Cell Transduction

T cell transduction was done as previously described (Maliar et al., Gastroenterology. 2012 November; 143(5):1375-1384. e5). Briefly, peripheral mouse blood lymphocytes (PBL) were isolated from the blood of wild-type C57BL/6 mice by density gradient centrifugation on Ficoll-Paque (Axis-shield). PBLs were activated in non-tissue culture-treated 6-well plates pre-coated with both mouse-anti-human CD3 (prepared in-house from hybridoma OKT3) and mouse-anti-human CD28 for 48 hours at 37° C. Activated lymphocytes were harvested, divided into two groups then co-cultured for 48 hours with 100 IU/ml IL-2 (untransduced cells) or for two consecutive retroviral transductions in RetroNectin (Takara Shuzu Ltd.) that was pre-coated to non-tissue culture-treated 6-well plates supplemented with 100 IU/ml human IL-2 (Novartis Pharma GMbH). At the end of transduction, both untransduced and transduced T cells were cultured in the presence of 350 IU/ml IL-2 for 24-72 hours for in-vitro or in-vivo assays, respectively. Transduction efficiency was monitored by flow cytometry analysis using FITC-mouse-anti-strep-tag IgG1 according to manufacturer instructions.

### Stimulation Assays

For glycan stimulation assay, 24 wells plate was coated with 6.25 μg/well HRP-conjugated streptavidin (SA-HRP; Jackson) diluted in 0.5 ml of 50 mM sodium carbonate-bicarbonate buffer, pH 9.5 (coating buffer) and incubated overnight at 4° C. The following day, unbound SA-HRP was washed twice with 1 ml PBS. Then, 1.5625 μg/well STn-Biotin or STn-PAA-Bio (Glycotech) in 0.5 ml PBS and plate was incubated for one more night at 4° C. The following day, unbound glycans were washed twice with 1 ml PBS. Then, 1 million cells/well of HuRA0 or UT mouse CAR-T in 1 ml RPMI+FCS+L-glu+P/S+beta mercaptoethanol+HEPES and plate was incubated for 16 hours at 37° C. The cell-free growth medium was collected and analyzed for IFN-γ, IL-2 and TNF-α production by ELISA using a mouse IFN-γ and TNF-α ELISA kit, according to the manufacturer's instructions (PeproTech).

For co-culturing stimulation assay, a total of 1×10⁶ untransduced or HuRA0 CAR transduced T cells were co-cultured with 0.5×10⁶ of cells (FaDu, Raji, Capan-2 or MEG-01) in 24-wells for 16 hours in a RPMI medium supplemented with 10% FCS, 2 mM glutamine and antibiotics. The cell-free growth medium was collected and analyzed for IFN-γ production by ELISA using a human IFN-γ ELISA kit, according to the manufacturer's instructions (R&D systems).

### Flow Cytometry

Cells were collected from plates using 10 mM EDTA. Cells were incubated with HuRA0 antibodies diluted in PBS+0.5% fish gelatin for 1 hour on ice, followed by incubation with Cy3 AffiniPure Goat Anti-Human IgG (H+L) (Jackson) diluted 1:100 in PBS+0.5% fish gelatin for 1 hour on ice. Fluorescence of cells were measured by CytoFLEX flow cytometry (Beckman Coulter).

### CDC Assay

For complement-dependent cytotoxicity (CDC) we used rabbit complement (Sigma). Cytotoxicity was evaluated by measuring lactate dehydrogenase (LDH) release using LDH Cytotoxicity Detection kit (Roche Applied Science) according to the manufacturer's instructions. All assays included maximum release control contains rabbit complement diluted 1:6 with 1% TritonX-100. For spontaneous release control, cells were incubated only with rabbit complement. Percentage cytotoxicity was calculated as follows: (test release-spontaneous release)/(maximum release-spontaneous release)×100. 2×10⁴ target Cells were incubated in triplicates with HuRA0 antibodies at 4 and 2 ng/μl for 1 hour on ice in 96-well round-bottom plates. Rabbit complement and triton were added and plates were incubated for 2 hours at 37° C. Then supernatants were collected and LDH release was determined.

### CAR T Glycan Specificity

5×10⁵ HuRA0-CAR T Cells and N29-CAR T Cells (served as irrelevant control CAR T cells) were incubated with 1 μM biotinylated-polyacrylamide conjugated glycans (Glycotech; 6-8 glycans per PAA-Bio molecule) diluted in PBS+0.5% fish gelatin (FACS buffer) for 45 minutes on ice, followed by incubation with APC-Streptavidin (Southern Biotech) diluted 1:1000 in FACS buffer for 30 minutes on ice. Cells were washed in FACS buffer and cell fluorescence was measured by CytoFLEX flow cytometry (Beckman Coulter).

### Adoptive Cell Transfer

C57BL/6 were maintained in a Specific Pathogen-Free Facility of the Tel Aviv University. 0.25×10⁶ B16F10 cells in 100 μl were injected subcutaneously into the flank of 6 to 8-week-old male mice. Two treatment regimens were evaluated for CAR T administration, at first regime, on day 10 mice were irradiated at 2Gy and on the following day, mice were adoptively transferred with 7×10⁶ T cells in 500 μl PBS untransduced or HuRA0 CAR T cells via intravenous injections. At the second regime, on day 3 mice were irradiated at 2Gy and on the following day, mice were adoptively transferred with 7×10⁶ T cells in 500 μl PBS untransduced or HuRA0 CAR T cells via intravenous injections. Tumor growth was monitored by a caliper every other day, and tumor volume calculated (tumor volume mm³=(length×width×depth)/2; n=5 per group).

### Statistical Analysis

Data were analyzed and graphed using Graphpad Prism V.8 (San Diego, CA, USA),

### Example 1. Humanization of mRA0 Antibodies

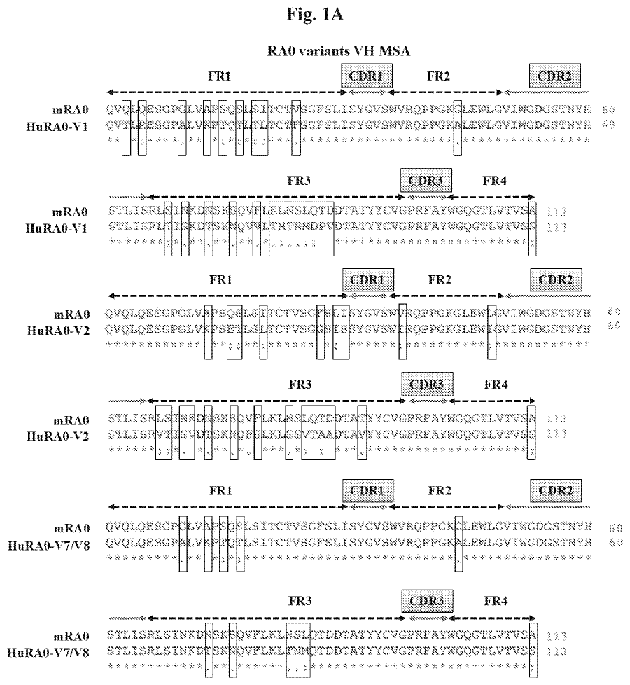

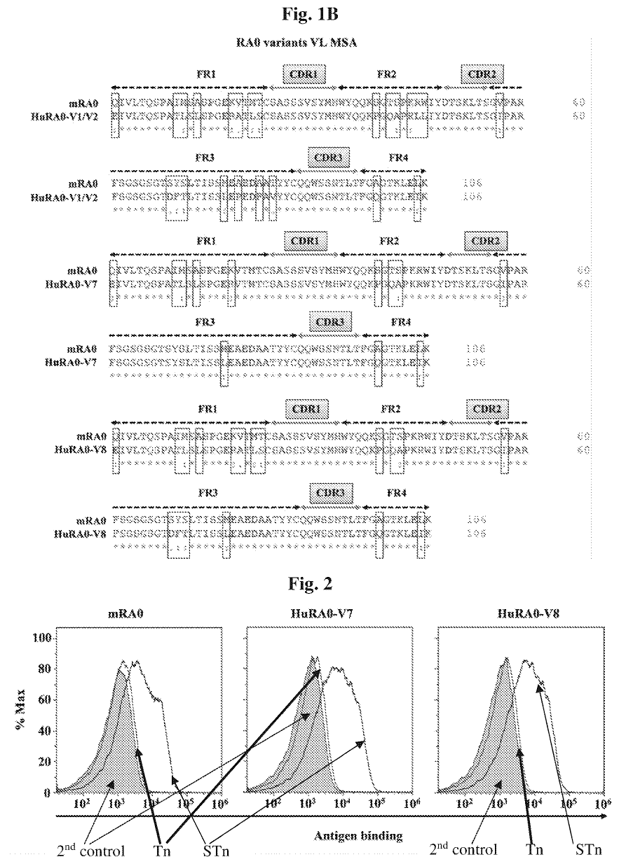

To reduce the immunogenicity of the mouse-derived native RA0 antibody, mutations to framework regions were introduced based on DNA sequence homology with human germline antibodies, to generate several humanized versions (HuRA0-V1, HuRA0-V2, HuRA0-V7 and HuRA0-V8). As described in the materials and methods sections, variants V7 and V8 have the same VH domain. Sequence alignment of these variants with mouse RA0 is presented in FIG. 1A (for VH domains) and FIG. 1B (for VL domains). The VH and VL of the mRA0 antibody have amino acid sequences as defined in SEQ ID NO: 1 and 2, respectively; the amino acid sequence of HV and VL of HuRA0-V1 are set forth in SEQ ID NO: 25 and 26, respectively; the amino acid sequence of HV and VL of HuRA0-V2 are set forth in SEQ ID NO: 27 and 26, respectively; the VH and VL of the HuRA0-V7 have amino acid sequences SEQ ID NO: 28 and 29, respectively, and the VH and VL of the HuRA0-V8 have amino acid sequences SEQ ID NO: 28 and 30, respectively.

### Example 2. Characterization of HuRA0 Antibodies

### Yeast Surface Display System

To characterize the properties of the humanized antibodies, the scFv fragments of mRA0, HuRA0-V1, HuRA0-V2, HuRA0-V7 (SEQ ID NO: 31) and HuRA0-V8 (SEQ ID NO: 32) were each cloned into yeast surface display system (YSD), followed by induction of their expression on the surface of these yeast cells. The binding of the yeast cells expressing the scFvs was examined by FACS. The results are presented in FIG. 2. While HuRA0-V1 and HuRA0-V2 did not bind STn or Tn antigens, the clones of scFv-mRA0 and scFv-HuRA0-V7 and HuRA0-V8 yeast variants show strong binding to the specific antigen STn, while no binding at all to the non-specific Tn antigen that lacks terminal sialic acid, and similar to the negative control staining. Therefore, further validation of specificity and affinity focused only on HuRA0-V7 and HuRA0-V8 variants using mRA0 as a control.

### Full-Length Antibodies

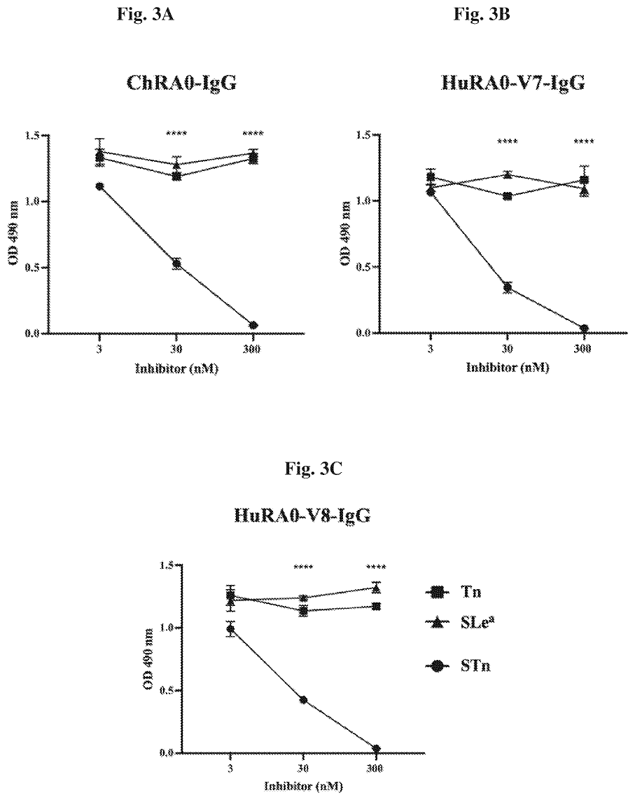

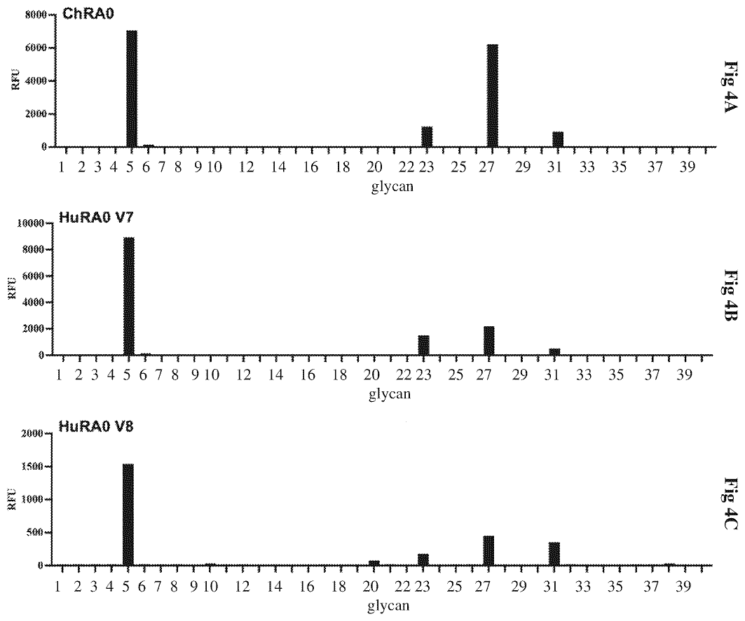

To further characterize the humanized variants as full-length antibodies, VH and VL sequences were cloned into human IgG p3BNC expression vectors by Gibson assembly (HuRA0-V7-hIgG, HuRA0-V8-hIgG; also referred as HuRA0-V7-IgG, HuRA0-V8-IgG). Similarly, VH and VL sequences of the mouse-derived antibodies were cloned into the same expression vectors to form chimeric antibodies (mRA0-hIgG; also referred as ChRA0-IgG). Full-length antibodies were produced by transfection of HEK-293A cells by polyethylenimine (PEI), then full-length antibodies were purified by protein A. To demonstrate the specificity of the three cloned antibodies, they were examined by ELISA against coated STn target (STn-PAA-Biotin) showing that competition with a soluble STn antigen (STn-PAA-Biotin) abrogated binding of antibodies, however competition with the non-sialylated target Tn (GalNAcα-R; Tn-PAA-Biotin) or the irrelevant sugar antigen sialyl-Lewis^{a} (SLe^{a}-PAA-Biotin) did not affect at all the binding of antibodies to the coated STn target antigen (FIG. 3). These data show that all the cloned antibodies (ChRA0-IgG, HuRA0-V7-IgG, HuRA0-V8-IgG) had specific reactivity against STn.

### Specificity to Glycan Microarray

Next, the high specificity full-length antibodies was also demonstrated by glycan microarrays showing strong binding to the STn glycan antigen (AcSTn; glycan #5) and to its 9-O-acetyated variant (9-O-GcSTn; glycan #23), as well as to its closely-related Neu5Ac-glycans (glycan #27, glycan #31), and to a lesser extent to its Neu5Gc-containing variant (GcSTn; glycan #6) and its 9-O-acetyated variant (9-O-GcSTn; glycan #24) (FIG. 4; examined at 0.019 ng/μl).

### Apparent K_{D}

Apparent K_{D} analysis on serial dilutions of the antibodies against the target glycans (by microarrays) showed that HuRA0-V7-IgG had an improved affinity against the top target antigens (AcSTn, glycan #5; 9-O-GcSTn, glycan #23) in comparison to the mouse antibody, while HuRA0-V8-IgG had slightly reduced affinity to these glycans, in particular to glycan #23 (Table 2). The slightly reduced affinity accompanied by higher specificity to AcSTn, glycan #5 antigen (FIG. 4) of HuRA0-V8 compared to ChRA0 can be beneficial for therapeutic approaches to reduce off-tumor toxicity as suggested e.g., by Ghorashian et al., (Nat Med. 2019 September; 25(9):1408-1414. doi: 10.1038/s41591-019-0549-5. Epub 2019 Sep. 2. PMID: 31477906).

TABLE 2
Apparent K_{D} measured on full length
antibodies in glycan microarray.

K_{D} (nM)

| Ab clone | Glycan #5 | Glycan #23 |
|---|---|---|
| ChRA0-IgG | 0.095 | 0.252 |
| HuRA0-V7-IgG | 0.049 | 0.193 |
| HuRA0-V8-IgG | 0.198 | 47.85 |

### Binding to Cancer Cells

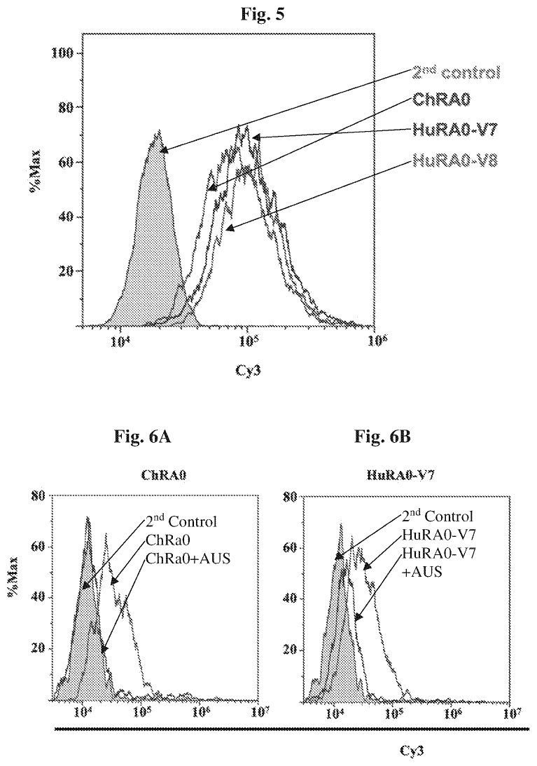

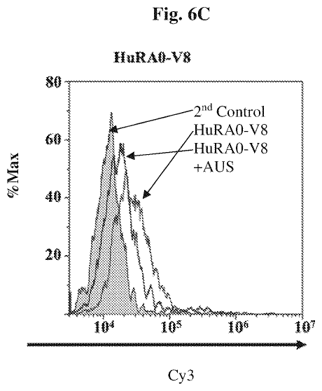

Analysis of antibody binding to cancer cells by FACS staining showed that the humanized antibodies (HuRA0-V7-IgG, HuRA0-V8-IgG) have strong binding to STn-positive B16F10 mouse melanoma cancer cell line, which was slightly improved compared to the mouse-derivative antibody clone ChRA0-IgG (FIG. 5). The binding of all antibody clones to this cancer cell line was inhibited by enzymatic removal of surface sialic acids by the sialidase Arthrobacter Ureafaciens Sialidase (AUS) (FIG. 6A-C), further supporting their specific recognition of the target also on specific cancer cells that express the STn antigen.

To evaluate the specificity of HuRA0 antibody against cancer tissues, human cancers tissue microarray (TMA) slides containing twenty-three different cancer tissues are stained by immunohistochemistry using biotinylated HuRA0 antibody (Bio-HuRA0-hIgG; prepared as described in methods section). The TMA include samples from melanoma, lung squamous cell carcinoma, lung adenocarcinoma, lung neuroendocrine cancer, papillary thyroid carcinoma, ductal breast carcinoma, Her-2 negative breast carcinoma, endometrial carcinoma, ovarian carcinoma, prostate adenocarcinoma, seminoma, hepatocellular carcinoma, renal clear cell carcinoma, diffuse type gastric adenocarcinoma, gastric GIST, pancreatic adenocarcinoma, colon adenocarcinoma, CLL/SLL lymphoma, follicular lymphoma, extranodal marginal zone lymphoma, mantle cell lymphoma, diffuse large B-cell lymphoma and lymphoblastic lymphoma. Of these tissues, endometrial carcinoma, ovarian carcinoma, prostate adenocarcinoma, seminoma, diffuse type gastric adenocarcinoma, pancreatic and colon adenocarcinomas show strong staining, lung adenocarcinoma and mantle cell lymphoma show moderate staining, and the other tissues seem to be negative for STn. These results demonstrate that several adenocarcinomas show very high level of staining, providing a clear indication that these types of cancer extensively express STn and may be targeted for treatment using the CAR of the present invention that binds specifically to STn antigen. Furthermore, HuRA0 antibody could efficiently bind mouse melanoma B16F10 cells, and facilitate dose-dependent complement-dependent cytotoxicity (CDC). Together, these results suggest that HuRA0 antibodies and their derivatives can be used to specifically target cancer cells for therapy, potentially also in vivo.

Altogether, these data indicate that humanized antibodies maintain high specificity, affinity and cell recognition characteristics as the original mouse-derived antibodies, or greater.

### Example 3. Reduced Immunogenicity of Humanized Antibody Clones

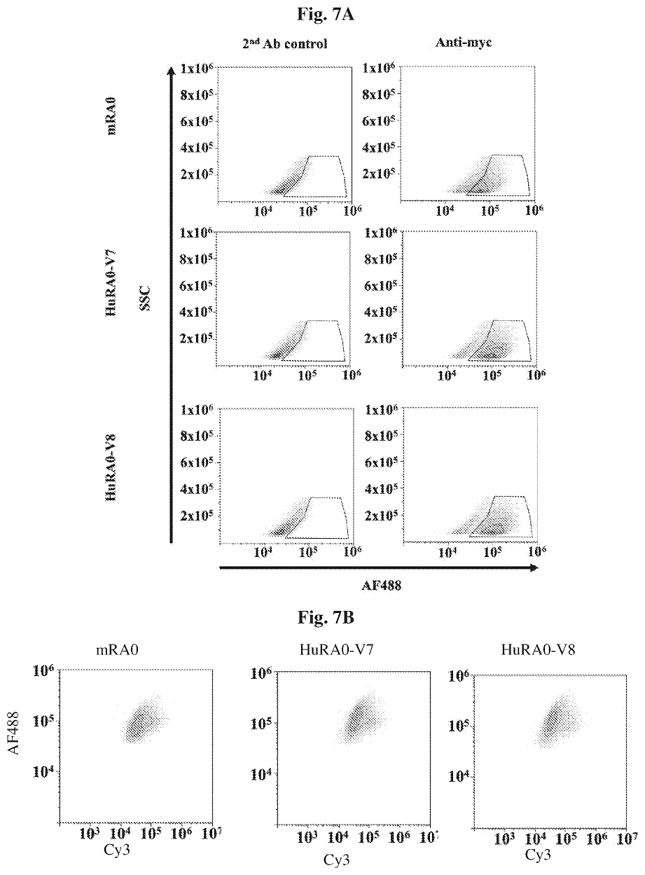

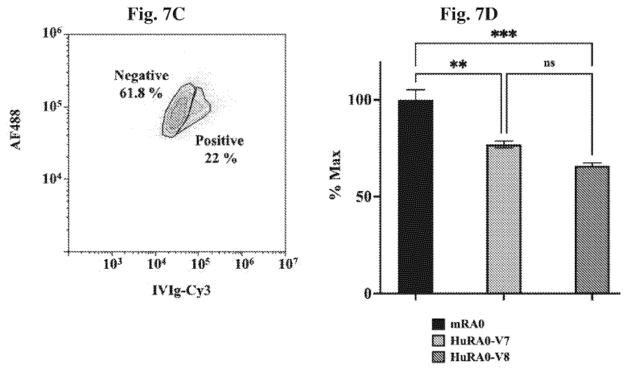

Immunogenicity of humanized antibody clones was evaluated by analysis of scFv recognition by pooled human IgG obtained from thousands of human donors (IVIg; Gamma Gard). For this purpose, IVIg was first pre-cleared from anti-yeast reactivity by serial incubations with yeast cells, then binding to scFv-expressing yeast cells was examined by FACS. The results are presented in FIG. 7.

Expression of scFv on yeast (mRA0-YSD, HuRA0-V7-YSD, HuRA0-V8-YSD) was examined by mouse-anti-c-Myc and pooled human IgG binding detected with anti-human IgG, and double positive labeling of scFv-expressing yeast cells was examined (FIG. 7A). The shape of the excessive IVIg-positive binding seemed to bulge out to the right clearly showing a separate population of the IVIg bound antibodies on the yeast cells (FIG. 7B), and the % of positive and negative IVIg labeling was determined by gating (FIG. 7C). In each clone, the percentage ratio of (% IVIg-positive cells/% IVIg-negative cells) was calculated for the three examined IVIg concentrations (25, 50 and 100 ng/μl), then averaged and normalized to mRA0 as the maximal signal. The results showed that HuRA0-V7 IVIg binding by IVIg was 25% lower compared to mRA0 IVIg binding, and HuRA0-V8 IVIg binding was 36% lower compared to mRA0 IVIg binding (FIG. 8D). The difference is statistically significant. Together, these data imply that the humanization process had a decreased recognition of the antibody fragments with a large collection of pooled human IgG.

In addition, it is also apparent that engineered chimeric antigen receptor T cells (CAR T), expressing the HuRA0-V7 or V8 scFv are expected to generate a lower immune reaction than the native mouse scFv. This is in turn would reduce the risk of cytokine storm or other immunological events. Therefore, the humanized antibodies have a great potential as therapeutic and diagnostic agents, with high specificity and affinity and less side effects of immune response against mouse-derived clones. Altogether, these data indicate that humanized antibodies maintain high specificity, affinity and cell recognition characteristics as the original mouse-derived antibody, or greater, and that HuRA0 antibodies and their derivatives can be used to specifically target cancer cells for therapy, potentially also in vivo.

### Example 4. CAR T Cells Bind STn and Induce In Vitro Cytotoxicity

To evaluate the efficacy of CAR-T therapy using an antigen-binding domain of HuRA0-V7 and V8 antibodies, a chimeric antigen receptor including these ABDs are synthesized. ScFv HuRA0-V7 and V8 are generated by linking the V_{H} and V_{L} domains of these antibodies by a 3×(GGGGS) spacer. The scFv is incorporated into a CAR backbone, containing a human CD28 transmembrane domain and intracellular co-stimulatory domain, followed by the FcγR ITAM intracellular signaling domain. The CAR may contain optionally a strep-tag connected through a 2×(GGGGS) spacer to CD28 the transmembrane domain. The sequence of the HuRA0-V7 and V8 CARs is as set forth in SEQ ID NOs: 41 and 42, respectively. The extracellular strep-tag allows to monitor CAR surface expression upon transduction. The CAR construct is cloned into the pMSGV1 retroviral vector, expressed in HEK 293T cells followed by generation of the PG-13 packaging cell line.

To demonstrate that the HuRA0-CAR construct maintains the high specificity against STn, the binding of HuRA0-V7 and V8-CARs or irrelevant-CAR to the specific STn-PAA-Bio glycan target or the non-specific Tn-PAA-Bio glycan target that lacks the sialic acid moiety is examined. FACS analysis demonstrate that while the irrelevant-N29-CAR did not bind any glycan target, HuRA0-CAR could bind STn, but not to the non-sialylated Tn antigen, supporting the specificity of HuRA0-CAR against the STn target antigen.

The ability of the glycans to facilitate CAR stimulation through cytokines secretion is then tested. In this assay. T cells expressing HuRA0-V7-CAR, HuRA0-V8-CAR and untransduced (UT) T cells that do not express a surface chimeric receptor are compared with glycan-presenting surfaces. For this purpose, we examine two different modes of glycans presentations and their effects on cytokine release: (1) condensed-rigid monovalent STn glycan with a terminal biotin at the non-reducing end (STn-Bio); and (2) dispersed and flexible polyvalent STn glycan conjugated at 6-8 copies onto a 30KDa polyacrylamide with a terminal biotin (STn-PAA-Bio), that better mimics the glycan presentation mode on the surface of cancer cells, as in mucins. While UT cells do not mediate any cytokine release, HuRA0-V7 and V8 CAR T cells are stimulated only with the polyvalent STn-PAA-Bio glycans and facilitate secretion of IFN-γ and TNF-α. These results support the efficacy of STn targeting by HuRA0-CAR T cells, particularly aiming STn glycans that are expressed closely to their natural presentation mode. Likewise, HuRA0-CAR T cells are stimulated by STn-expressing-MEG-01 acute myeloid leukemia AML cells resulting in IFN-γ cytokine secretion.

### Example 5. HuRA0-CAR T Cells Inhibit Tumor Growth In Vivo in Mice

To assess the cytotoxicity of HuRA0-CAR T cells in vivo, several different types of cancer cells, including endometrial carcinoma cells, breast cancer cells (both HER2 positive and negative), ovarian carcinoma cells, prostate adenocarcinoma cells, seminoma cells, diffuse type gastric adenocarcinoma cells, pancreatic cancer cells, colon adenocarcinoma cells and B16F10 melanoma cells are subcutaneously injected into the flank of C57BL/6 mice. Once tumors are palpable, the mice are irradiated, and then treated by a single intravenous dose of HuRA0-V7 CAR T cells HuRA0-V8 CAR T cells or untransduced T cells. It is shown that a single administration of HuRA0 CAR T cells lead to a significant inhibition of tumor growth compared to the control group (treated with untransduced T cells). It can be seen that the mean volume of tumors in mice treated with HuRA0-CAR T cells is at least 2 times smaller than in the control group treated with UT T cells. Together, these results demonstrate the therapeutic potential of HuRA0-CAR against STn-expressing cancer cells. Considering lower, it is estimated that this treatment leads to lower immune response and specifically prevents cytokine storm. The evaluation of two different regimens demonstrate the high potency of the treatment, which might be suitable for different cancer stages, as CAR T administration in day 4 or 11 show tumor growth inhibition. Given that 80% of human carcinoma express STn, this could provide robust novel immunotherapy approach.

Although the present invention has been described herein above by way of preferred embodiments thereof, it can be modified, without departing from the spirit and nature of the subject invention.

1-52. (canceled)

53. A humanized monoclonal antibody (mAb) or a fragment thereof that specifically binds to Sialyl Tn (STn) glycan, comprising an antigen-binding domain comprising a heavy-chain variable domain (VH) and a light-chain variable domain (VL), wherein the VH and the VL each comprises three complementarity determining regions (CDRs) and four framework regions (FR), wherein the VH domain comprises amino acid sequence SEQ ID NO:1 in which from 9 to 16 amino acids in the framework regions are substituted and the VL domain comprises amino acid sequence SEQ ID NO:2 in which from 9 to 20 amino acids in the framework regions are substituted.

54. The humanized mAb or the fragment according to claim 53, characterized by at least one of:
(i) wherein the VH-CDRs 1, 2 and 3 comprise amino acid sequences SEQ ID NOs: 3, 4 and 5, respectively, and the VL-CDRs 1, 2, and 3 comprise amino acid sequences SEQ ID NOs: 6, 7, and 8, respectively;
(ii) at least 9 substitutions in VH domain are at positions selected from positions 10, 13, 15, 17, 44, 73, 76, 83, 84, 85 and 113 of SEQ ID NO: 1;
(iii) all amino acids at positions 10, 13, 15, 17, 44, 73, 76, 83, 84, 85 and 113 of SEQ ID NO: 1 are substituted;
(iv) at least 9 substitutions in the VL domain are at positions selected from positions 1, 10, 11, 13, 18, 19, 21, 22, 39, 41, 42, 57, 69, 70, 71, 77, 99 and 105 of SEQ ID NO: 2; or
(v) all amino acids at positions 1, 10, 11, 13, 18, 39, 41, 42, 57, 77, 99 and 105 of SEQ ID NO: 2 are substituted or wherein all amino acids at positions 1, 10, 11, 13, 18, 19, 21, 22, 39, 41, 42, 57, 69, 70, 71, 77, 99 and 105 of SEQ ID NO: 2 are substituted.

55. The humanized mAb or the fragment according to claim 54, characterized by one of: (i) the VH domain comprises or consists of amino acid sequence SEQ ID NO: 28, (ii) the VL domain comprises or consists of amino acid sequence SEQ ID NO: 29, (iii) VL domain comprises or consists of amino acid sequence SEQ ID NO: 30; and (iv) the VH domain comprises or consists of amino acid sequence SEQ ID NO: 28 and the VL domain comprises or consists of an amino acid sequence selected from SEQ ID NO: 29 and 30.

56. The humanized mAb or the fragment according to 53, characterized by one of:
(i) said antibody or fragment binds STn glycan with an equilibrium dissociation constant (KD) of from about 0.01 to about 30 nM or from 0.02 to 0.5 nM;
(ii) wherein the fragment is a single chain variable fragment (scFv);
(iii) wherein the fragment is a single chain variable fragment (scFv) comprising an amino acid sequence selected from SEQ ID NO: 31 and 32.

57. A chimeric antigen receptor (CAR) comprising the humanized mAb or the fragment thereof according to claim 53.

58. The CAR according to claim 57, comprising the fragment of the humanized mAb and wherein the fragment is a single chain variable fragment (scFv) comprising an amino acid sequence selected from SEQ ID NO: 31 and 32.

59. The CAR according to claim 57, wherein the CAR comprises a transmembrane domain (TM domain), one or more costimulatory domains and an activation domain.

60. The CAR according to claim 59, characterized by at least one of:
(i) the TM domain is a TM domain of a receptor selected from CD28 and CD8;
(ii) the costimulatory domain is selected from a costimulatory domain of a protein selected from CD28, 4-1BB, OX40, iCOS, CD27, CD80, and CD70;
(iii) the antigen-binding domain is linked to the TM domain via a spacer;
(iv) the activation domain is selected from FcRγ and CD3-ζ activation domains;
(v) further comprising a leading peptide.

61. The CAR according to claim 60, comprising an amino acid sequence selected from SEQ ID NO: 41, 42, 48 and 49.

62. A conjugate comprising the humanized mAb or fragment thereof according to claim 53.

63. A nucleic acid molecule encoding at least one chain of the humanized monoclonal antibody or fragment thereof according to claim 53 or a CAR comprising the humanized monoclonal antibody or fragment thereof.

64. The nucleic acid molecule according to claim 63, encoding an amino acid sequence selected from SEQ ID NO: 28, 29, 30, 31, 32, a combination of SEQ ID NO: 28 and 29, and a combination of SEQ ID NO: 28 and 30.

65. The nucleic acid molecule according to claim 63, comprising a nucleic acid sequence selected from the group consisting of SEQ ID NO: 33, 34, 35, 45, 36, 37, 41, 42, 50, 51 and a combination thereof.

66. A cell comprising the humanized monoclonal antibody or the antibody fragment according to claim 53, the CAR comprising same or the nucleic acid molecule encoding the humanized monoclonal antibody, the antibody fragment or the CAR.

67. The cell according to claim 66, wherein the cell is characterized by at least one of:
(i) the cell expresses or is capable of expressing the CAR;
(ii) the cell is selected from a T cell and a natural killer (NK) cell;
(iii) the cell comprises the CAR;
(iv) the cell comprises a nucleic acid molecule encoding the CAR.

68. A composition comprising the humanized monoclonal antibodies or fragments thereof according to claim 53 or the CAR comprising same or a plurality of cells comprising the humanized monoclonal antibodies, fragments thereof or the CAR, and a carrier.

69. The composition according to claim 68, wherein the composition is selected from the group consisting of a diagnostic composition, and a pharmaceutical composition comprising a pharmaceutically acceptable carrier.

70. A method for treating cancer in a subject in need thereof comprising administering to said subject a therapeutically effective amount of the monoclonal antibodies or fragments thereof according to claim 53, the conjugates thereof, the CAR comprising the monoclonal antibodies or fragment thereof, the cells comprising the monoclonal antibodies, the fragments thereof or the CAR or the pharmaceutical composition comprising any one of the above.

71. A method for detecting the presence or for quantifying STn glycan in a biological sample, the method comprises contacting a biological sample with the monoclonal antibodies or antibody fragments according to claim 53 or the conjugates comprising same and detecting the presence or assessing the amount of STn glycan in the sample.

72. A kit for diagnosing a cancer in a subject or for quantifying STn glycan in a biological sample, wherein the kit comprises monoclonal antibodies or antibody fragments according to claim 53 or a conjugate comprising same and means for detecting the amount of the antibodies or antibody fragments bound to cells or to STn glycan in the biological sample.


SEQUENCE LISTING (51 sequences)

SEQ ID NO: 1 - protein, 113 aa, Artificial Sequence
  Synthesized sequence
QVQLQESGPGLVAPSQSLSITCTVSGFSLISYGVSWVRQPPGKGLEWLGVIWGDGSTNYHSTLISRLSINKDNSKSQVFLKLNSLQTDDTATYYCVGPRFAYWGQGTLVTVSA

SEQ ID NO: 2 - protein, 106 aa, Artificial Sequence
  Synthesized sequence
QIVLTQSPAIMSASPGEKVTMTCSASSSVSYMHWYQQKSGTSPKRWIYDTSKLTSGVPARFSGSGSGTSYSLTISSMEAEDAATYYCQQWSSNTLTFGAGTKLELK

SEQ ID NO: 3 - protein, 5 aa, Artificial Sequence
  Synthesized sequence
SYGVS

SEQ ID NO: 4 - protein, 16 aa, Artificial Sequence
  Synthesized sequence
VIWGDGSTNYHSTLIS

SEQ ID NO: 5 - protein, 5 aa, Artificial Sequence
  Synthesized sequence
PRFAY

SEQ ID NO: 6 - protein, 10 aa, Artificial Sequence
  Synthesized sequence
SASSSVSYMH

SEQ ID NO: 7 - protein, 7 aa, Artificial Sequence
  Synthesized sequence
DTSKLTS

SEQ ID NO: 8 - protein, 9 aa, Artificial Sequence
  Synthesized sequence
QQWSSNTLT

SEQ ID NO: 9 - protein, 5 aa, Artificial Sequence
  Synthesized sequence
GGGGS

SEQ ID NO: 10 - protein, 15 aa, Artificial Sequence
  Synthesized sequence
GGGGSGGGGSGGGGS

SEQ ID NO: 11 - protein, 10 aa, Artificial Sequence
  Synthesized sequence
GGGGSGGGGS

SEQ ID NO: 12 - protein, 40 aa, Artificial Sequence
  Synthesized sequence
SKRSRLLHSDYMNMTPRRPGPTRKHYQPYAPPRDFAAYRS

SEQ ID NO: 13 - protein, 110 aa, Artificial Sequence
  Synthesized sequence
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 14 - protein, 38 aa, Artificial Sequence
  Synthesized sequence
QVRKAAITSYEKSDGVYTGLSTRNQETYETLKHEKPPQ

SEQ ID NO: 15 - protein, 22 aa, Artificial Sequence
  Synthesized sequence
MDFQVQIFSFLLISASVIMSRG

SEQ ID NO: 16 - DNA, 339 bp, Artificial Sequence
  Synthesized sequence
caggtgcagctgcaggagtctggacctggcctggtggcgccctcacagagcctgtccatcacatgcactgtctcagggttctcattaatcagctatggtgtaagctgggttcgccagcctccaggaaagggtctggagtggctgggagtaatatggggtgacgggagcacaaattatcattcaactctcatatccagactgagcatcaacaaggataactccaagagccaagttttcttaaaactgaacagtctgcaaactgatgacacagccacgtactactgtgtcggaccccggtttgcttactggggccaagggactctggtcactgtctctgca

SEQ ID NO: 17 - DNA, 318 bp, Artificial Sequence
  Synthesized sequence
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgacttctggagtccctgctcgcttcagtggcagtgggtctgggacctcttactctctcacaatcagcagcatggaggctgaagatgctgccacttattactgccagcagtggagtagtaatacgctcacgttcggtgctgggaccaagctggagctgaaa

SEQ ID NO: 18 - DNA, 45 bp, Artificial Sequence
  Synthesized sequence
ggaggtggcggtagcggaggcggcggttctggaggtggcgggagc

SEQ ID NO: 19 - DNA, 15 bp, Artificial Sequence
  Synthesized sequence
ggaggtggcggtagc

SEQ ID NO: 20 - DNA, 30 bp, Artificial Sequence
  Synthesized sequence
ggaggtggcggtagcggaggcggcggttct

SEQ ID NO: 21 - DNA, 60 bp, Artificial Sequence
  Synthesized sequence
ggaggtggcggtagcggaggcggcggttctggaggtggcgggagcggaggcggcggttct

SEQ ID NO: 22 - DNA, 330 bp, Artificial Sequence
  Synthesized sequence
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatct

SEQ ID NO: 23 - DNA, 117 bp, Artificial Sequence
  Synthesized sequence
caagtgcgaaaggcagctataaccagctatgagaaatcagatggtgtttacacgggcctgagcaccaggaaccaggagacttacgagactctgaagcatgagaaaccaccacagtag

SEQ ID NO: 24 - DNA, 66 bp, Artificial Sequence
  Synthesized sequence
atggattttcaggtgcagattttcagcttcctgctaatcagtgcctcagtcataatgtccagagga

SEQ ID NO: 25 - protein, 113 aa, Artificial Sequence
  Synthesized sequence
QVTLRESGPALVKPTQTLTLTCTFSGFSLISYGVSWVRQPPGKALEWLGVIWGDGSTNYHSTLISRLTISKDTSKNQVVLTMTNMDPVDTATYYCVGPRFAYWGQGTLVTVSS

SEQ ID NO: 26 - protein, 106 aa, Artificial Sequence
  Synthesized sequence
EIVLTQSPATLSLSPGERATLSCSASSSVSYMHWYQQKPGQAPRLLIYDTSKLTSGIPARFSGSGSGTDFTLTISSLEPEDFAVYYCQQWSSNTLTFGQGTKLEIK

SEQ ID NO: 27 - protein, 113 aa, Artificial Sequence
  Synthesized sequence
QVQLQESGPGLVKPSETLSLTCTVSGGSISSYGVSWIRQPPGKGLEWIGVIWGDGSTNYHSTLISRVTISVDTSKNQFSLKLSSVTAADTAVYYCVGPRFAYWGQGTLVTVSS

SEQ ID NO: 28 - protein, 113 aa, Artificial Sequence
  Synthesized sequence
QVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSS

SEQ ID NO: 29 - protein, 106 aa, Artificial Sequence
  Synthesized sequence
EIVLTQSPATLSLSPGERVTMTCSASSSVSYMHWYQQKPGQAPKRWIYDTSKLTSGIPARFSGSGSGTSYSLTISSLEAEDAATYYCQQWSSNTLTFGQGTKLEIK

SEQ ID NO: 30 - protein, 106 aa, Artificial Sequence
  Synthesized sequence
EIVLTQSPATLSLSPGERATLSCSASSSVSYMHWYQQKPGQAPKRWIYDTSKLTSGIPARFSGSGSGTDFTLTISSLEAEDAATYYCQQWSSNTLTFGQGTKLEIK

SEQ ID NO: 31 - protein, 234 aa, Artificial Sequence
  Synthesized sequence
QVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERVTMTCSASSSVSYMHWYQQKPGQAPKRWIYDTSKLTSGIPARFSGSGSGTSYSLTISSLEAEDAATYYCQQWSSNTLTFGQGTKLEIK

SEQ ID NO: 32 - protein, 234 aa, Artificial Sequence
  Synthesized sequence
QVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERATLSCSASSSVSYMHWYQQKPGQAPKRWIYDTSKLTSGIPARFSGSGSGTDFTLTISSLEAEDAATYYCQQWSSNTLTFGQGTKLEIK

SEQ ID NO: 33 - protein, 339 aa, Artificial Sequence
  Synthesized sequence
CAGGTACAACTTCAGGAGAGCGGGCCTGCTCTCGTGAAACCCACGCAAACTCTTTCTATTACTTGCACGGTCTCAGGTTTTTCTCTGATTTCATACGGTGTCAGTTGGGTAAGACAACCCCCGGGTAAAGCGTTGGAGTGGTTGGGAGTAATTTGGGGCGACGGGTCTACGAATTACCACTCTACACTCATATCAAGACTGTCCATCAATAAAGATACCTCTAAAAATCAAGTATTTTTGAAACTGACCAACATGCAGACAGATGATACAGCCACATATTACTGTGTCGGGCCGAGGTTTGCGTATTGGGGGCAAGGTACATTGGTTACCGTTAGTTCA

SEQ ID NO: 34 - DNA, 318 bp, Artificial Sequence
  Synthesized sequence
gaaattgtgctgacccaatccccagccactctcagtctctcacccggcgaaagagtaacaatgacttgtagtgcgtccagctccgtttcttacatgcactggtatcagcagaaaccaggccaggccccgaaaaggtggatatatgatacctctaaactgacctctggcattcccgcccgctttagtggctctggctcaggtacatcatacagtttgactatatcctctcttgaggcagaagacgctgcgacatattattgccaacaatggtctagcaatacgctcaccttcggccaagggacaaaactggaaattaaa

SEQ ID NO: 35 - DNA, 318 bp, Artificial Sequence
  Synthesized sequence
gaaattgtactgactcagtctccagctactttgtctctctcccctggggagcgggctacgctctcttgctcagcttcaagtagcgtgtcctatatgcactggtatcagcagaagccagggcaggccccgaaaagatggatttatgacaccagtaaacttacatccggaatccccgcgcgattctctggaagcggctcaggaaccgactttacactcacgatttctagcttggaagcagaagacgctgcgacctactattgccaacaatggtcttccaacacgctcacgtttggtcaaggaaccaaactggagataaaa

SEQ ID NO: 36 - DNA, 702 bp, Artificial Sequence
  Synthesized sequence
caggtgcaactgcaagagagtggcccagcccttgttaaacccacacagactctcagtattacttgtacagtatccggcttcagtctcatcagttacggggtatcctgggtgcgacaaccaccgggtaaagcccttgaatggctcggcgttatatggggggacggctctacgaattatcatagcaccttgatttcccgccttagtataaataaggatacaagcaaaaatcaagtgttcttgaaacttacgaatatgcagactgacgatacggcgacctactactgtgtaggccctcgattcgcgtactggggacagggaacattggttactgtttcatcaggaggtggcggtagcggaggcggcggttctggaggtggcgggagcgaaattgtgctgacccaatccccagccactctcagtctctcacccggcgaaagagtaacaatgacttgtagtgcgtccagctccgtttcttacatgcactggtatcagcagaaaccaggccaggccccgaaaaggtggatatatgatacctctaaactgacctctggcattcccgcccgctttagtggctctggctcaggtacatcatacagtttgactatatcctctcttgaggcagaagacgctgcgacatattattgccaacaatggtctagcaatacgctcaccttcggccaagggacaaaactggaaattaaa

SEQ ID NO: 37 - DNA, 702 bp, Artificial Sequence
  Synthesized sequence
caggtacaacttcaggagagcgggcctgctctcgtgaaacccacgcaaactctttctattacttgcacggtctcaggtttttctctgatttcatacggtgtcagttgggtaagacaacccccgggtaaagcgttggagtggttgggagtaatttggggcgacgggtctacgaattaccactctacactcatatcaagactgtccatcaataaagatacctctaaaaatcaagtatttttgaaactgaccaacatgcagacagatgatacagccacatattactgtgtcgggccgaggtttgcgtattgggggcaaggtacattggttaccgttagttcaggaggtggcggtagcggaggcggcggttctggaggtggcgggagcgaaattgtactgactcagtctccagctactttgtctctctcccctggggagcgggctacgctctcttgctcagcttcaagtagcgtgtcctatatgcactggtatcagcagaagccagggcaggccccgaaaagatggatttatgacaccagtaaacttacatccggaatccccgcgcgattctctggaagcggctcaggaaccgactttacactcacgatttctagcttggaagcagaagacgctgcgacctactattgccaacaatggtcttccaacacgctcacgtttggtcaaggaaccaaactggagataaaa

SEQ ID NO: 38 - DNA, 339 bp, Artificial Sequence
  Synthesized sequence
caggtcaccttgagggagtctggtcctgcgctggtgaaacccacacagaccctcaccctgacctgcaccttctctgggttctcactcatcagctatggtgtaagctgggtccgtcagcccccagggaaggccctggagtggcttggagtaatatggggtgacgggagcacaaattatcattcaactctcatatccaggctcaccatctccaaggacacctccaaaaaccaggtggtccttacaatgaccaacatggaccctgtggacacagccacgtattactgtgtcggaccccggtttgcttactggggccagggcaccctggtcaccgtctcctca

SEQ ID NO: 39 - DNA, 339 bp, Artificial Sequence
  Synthesized sequence
caggtgcagctgcaggagtcgggcccaggactggtgaagccttcggagaccctgtccctcacctgcactgtctctggtggctccatcagtagctatggtgtaagctggatccggcagcccccagggaagggactggagtggattggggtaatatggggtgacgggagcacaaattatcattcaactctcatatcccgagtcaccatatccgtagacacgtccaagaaccagttctccctgaagctgagctctgtgaccgccgcagacacggccgtgtattactgtgtcggaccccggtttgcttactggggccagggcaccctggtcaccgtctcctca

SEQ ID NO: 40 - DNA, 318 bp, Artificial Sequence
  Synthesized sequence
gaaattgtgttgacacagtctccagccaccctgtctttgtctccaggggaaagagccaccctctcctgcagtgccagctcaagtgtaagttacatgcactggtaccaacagaaacctggccaggctcccaggctcctcatctatgacacatccaaactgacttctggcatcccagccaggttcagtggcagtgggtctgggacagacttcactctcaccatcagcagcctagagcctgaagattttgcagtttattactgtcagcagtggagtagtaatacgctcacgtttggccaggggaccaagctggagatcaaa

SEQ ID NO: 41 - protein, 414 aa, Artificial Sequence
  Synthesized sequence
MDFQVQIFSFLLISASVIMSRGQVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERVTMTCSASSSVSYMHWYQQKPGQAPKRWIYDTSKLTSGIPARFSGSGSGTSYSLTISSLEAEDAATYYCQQWSSNTLTFGQGTKLEIKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 42 - protein, 414 aa, Artificial Sequence
  Synthesized sequence
MDFQVQIFSFLLISASVIMSRGQVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERATLSCSASSSVSYMHWYQQKPGQAPKRWIYDTSKLTSGIPARFSGSGSGTDFTLTISSLEAEDAATYYCQQWSSNTLTFGQGTKLEIKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 43 - DNA, 1245 bp, Artificial Sequence
  Synthesized sequence
atggattttcaggtgcagattttcagcttcctgctaatcagtgcctcagtcataatgtccagaggacaggtgcaactgcaagagagtggcccagcccttgttaaacccacacagactctcagtattacttgtacagtatccggcttcagtctcatcagttacggggtatcctgggtgcgacaaccaccgggtaaagcccttgaatggctcggcgttatatggggggacggctctacgaattatcatagcaccttgatttcccgccttagtataaataaggatacaagcaaaaatcaagtgttcttgaaacttacgaatatgcagactgacgatacggcgacctactactgtgtaggccctcgattcgcgtactggggacagggaacattggttactgtttcatcaggaggtggcggtagcggaggcggcggttctggaggtggcgggagcgaaattgtgctgacccaatccccagccactctcagtctctcacccggcgaaagagtaacaatgacttgtagtgcgtccagctccgtttcttacatgcactggtatcagcagaaaccaggccaggccccgaaaaggtggatatatgatacctctaaactgacctctggcattcccgcccgctttagtggctctggctcaggtacatcatacagtttgactatatcctctcttgaggcagaagacgctgcgacatattattgccaacaatggtctagcaatacgctcaccttcggccaagggacaaaactggaaattaaaggaggtggcggtagcggaggcggcggttctgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 44 - DNA, 1245 bp, Artificial Sequence
  Synthesized sequence
atggattttcaggtgcagattttcagcttcctgctaatcagtgcctcagtcataatgtccagaggacaggtacaacttcaggagagcgggcctgctctcgtgaaacccacgcaaactctttctattacttgcacggtctcaggtttttctctgatttcatacggtgtcagttgggtaagacaacccccgggtaaagcgttggagtggttgggagtaatttggggcgacgggtctacgaattaccactctacactcatatcaagactgtccatcaataaagatacctctaaaaatcaagtatttttgaaactgaccaacatgcagacagatgatacagccacatattactgtgtcgggccgaggtttgcgtattgggggcaaggtacattggttaccgttagttcaggaggtggcggtagcggaggcggcggttctggaggtggcgggagcgaaattgtactgactcagtctccagctactttgtctctctcccctggggagcgggctacgctctcttgctcagcttcaagtagcgtgtcctatatgcactggtatcagcagaagccagggcaggccccgaaaagatggatttatgacaccagtaaacttacatccggaatccccgcgcgattctctggaagcggctcaggaaccgactttacactcacgatttctagcttggaagcagaagacgctgcgacctactattgccaacaatggtcttccaacacgctcacgtttggtcaaggaaccaaactggagataaaaggaggtggcggtagcggaggcggcggttctgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 45 - DNA, 339 bp, Artificial Sequence
  Synthesized sequence
caggtgcaactgcaagagagtggcccagcccttgttaaacccacacagactctcagtattacttgtacagtatccggcttcagtctcatcagttacggggtatcctgggtgcgacaaccaccgggtaaagcccttgaatggctcggcgttatatggggggacggctctacgaattatcatagcaccttgatttcccgccttagtataaataaggatacaagcaaaaatcaagtgttcttgaaacttacgaatatgcagactgacgatacggcgacctactactgtgtaggccctcgattcgcgtactggggacagggaacattggttactgtttcatca

SEQ ID NO: 46 - protein, 8 aa, Artificial Sequence
  Synthesized sequence
WSHPQFEK

SEQ ID NO: 47 - DNA, 24 bp, Artificial Sequence
  Synthesized sequence
tggagccacccgcagttcgagaaa

SEQ ID NO: 48 - protein, 422 aa, Artificial Sequence
  Synthesized sequence
MDFQVQIFSFLLISASVIMSRGQVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERVTMTCSASSSVSYMHWYQQKPGQAPKRWIYDTSKLTSGIPARFSGSGSGTSYSLTISSLEAEDAATYYCQQWSSNTLTFGQGTKLEIKWSHPQFEKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 49 - protein, 422 aa, Artificial Sequence
  Synthesized sequence
MDFQVQIFSFLLISASVIMSRGQVQLQESGPALVKPTQTLSITCTVSGFSLISYGVSWVRQPPGKALEWLGVIWGDGSTNYHSTLISRLSINKDTSKNQVFLKLTNMQTDDTATYYCVGPRFAYWGQGTLVTVSSGGGGSGGGGSGGGGSEIVLTQSPATLSLSPGERATLSCSASSSVSYMHWYQQKPGQAPKRWIYDTSKLTSGIPARFSGSGSGTDFTLTISSLEAEDAATYYCQQWSSNTLTFGQGTKLEIKWSHPQFEKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 50 - DNA, 1269 bp, Artificial Sequence
  Synthesized sequence
atggattttcaggtgcagattttcagcttcctgctaatcagtgcctcagtcataatgtccagaggacaggtgcaactgcaagagagtggcccagcccttgttaaacccacacagactctcagtattacttgtacagtatccggcttcagtctcatcagttacggggtatcctgggtgcgacaaccaccgggtaaagcccttgaatggctcggcgttatatggggggacggctctacgaattatcatagcaccttgatttcccgccttagtataaataaggatacaagcaaaaatcaagtgttcttgaaacttacgaatatgcagactgacgatacggcgacctactactgtgtaggccctcgattcgcgtactggggacagggaacattggttactgtttcatcaggaggtggcggtagcggaggcggcggttctggaggtggcgggagcgaaattgtgctgacccaatccccagccactctcagtctctcacccggcgaaagagtaacaatgacttgtagtgcgtccagctccgtttcttacatgcactggtatcagcagaaaccaggccaggccccgaaaaggtggatatatgatacctctaaactgacctctggcattcccgcccgctttagtggctctggctcaggtacatcatacagtttgactatatcctctcttgaggcagaagacgctgcgacatattattgccaacaatggtctagcaatacgctcaccttcggccaagggacaaaactggaaattaaatggagccacccgcagttcgagaaaggaggtggcggtagcggaggcggcggttctgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 51 - DNA, 1269 bp, Artificial Sequence
  Synthesized sequence
atggattttcaggtgcagattttcagcttcctgctaatcagtgcctcagtcataatgtccagaggacaggtacaacttcaggagagcgggcctgctctcgtgaaacccacgcaaactctttctattacttgcacggtctcaggtttttctctgatttcatacggtgtcagttgggtaagacaacccccgggtaaagcgttggagtggttgggagtaatttggggcgacgggtctacgaattaccactctacactcatatcaagactgtccatcaataaagatacctctaaaaatcaagtatttttgaaactgaccaacatgcagacagatgatacagccacatattactgtgtcgggccgaggtttgcgtattgggggcaaggtacattggttaccgttagttcaggaggtggcggtagcggaggcggcggttctggaggtggcgggagcgaaattgtactgactcagtctccagctactttgtctctctcccctggggagcgggctacgctctcttgctcagcttcaagtagcgtgtcctatatgcactggtatcagcagaagccagggcaggccccgaaaagatggatttatgacaccagtaaacttacatccggaatccccgcgcgattctctggaagcggctcaggaaccgactttacactcacgatttctagcttggaagcagaagacgctgcgacctactattgccaacaatggtcttccaacacgctcacgtttggtcaaggaaccaaactggagataaaatggagccacccgcagttcgagaaaggaggtggcggtagcggaggcggcggttctgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag